SEQUENCE LISTING (33 sequences)

SEQ ID NO: 1 - DNA, 1099 bp, Oryzias latipes
  misc_feature: cDNA for gene 1
gtaaacggacagctagagttcagctgaaaagaagtgtaattctattcggacgtagtctttaaaaaaaaaatgaaggtgccagaggcggaattaatgagcgacattctgaagaggctgacgggagagtctgctctgccgctgtactgctgcatcgagaagtacaagcgcgagaggaacggcctctactttgtcgccgaggatttcactgaaaccgtcaaaaaaagagaaatggtcaacgccaaggaaagactgagaataaggaacttgaacacaatgttctcccgactgaagcgcatgctgcctctaatgcaaccagacaaaaagccgagtaaagttgatacactcaaagcagccactgaatacattcgacttcttcttgctgttttgcgggacactgaaaataacaacactgggacggattttctaaagaatgcaatcacttatggtcagcaggatggcttcgccaatgacctctggagaatggacgatttcttgaacctgtcagatgatcacatggaggatgggttcaccatgccagcagaacctgcagcagaggatggagacatgactagactggtgttgcagcattgtgtgatgcctgcataccagttcatcatccaagtagcgcccgatcaagcttcgagggattaattagccaccgcctcccgactgcacatcccaaccactgacccgatgtccttgctatcttggacattgatgacttgcactctttttccttgacacttattataaaatggcttgatttaaaacctaacccttaatttttttttcattatttagttgtacatacattggaagttgatgcatctagtaaacatacctaaaaagaactgctgctctaagagttctcattttgctttcagctgtactctatttgaggaaatgacttaatttatgtatttttcttgtaattgtaatcataaagccccaatgaaaaagatgcaaatttgtgacaatttgttgaggtcaatgtgatctaagttgtcaatatgaattatctgtttgaaaacttgaatctaatttaaattcagaaatgtctaaatgtgtttagttaatgatcaaataaacaagctttaag

SEQ ID NO: 2 - DNA, 938 bp, Oryzias latipes
  misc_feature: cDNA for gene 2
gaccgccgtagtggtgtccacttcgattcctggaaacctcgaggaaaaaagtgaaataagcaacggaaagatcgggaatcctgaggcttgcatcaaacatcccttacagaacaggtggactctttggtttttcaagaatgacaaaagcaaaacctggcaggccaaccttcgtctcatctctacgtttgacacagttgaggatttctgggcgttatacaatcatattcaactgtcaagtaacttgatgtctggctgtgactactctctgtttaaggatgggattgaacccatgtgggaggatgaaaggaatcggcgtgggggccgctggctcatcaccctgtccaaacatcaacggaaaatggatctggaccggttctggttggagacgcttttgtgtctcgtgggcgaagcctttgatgatcacagcgatgatgtttgtggagccgtcatcaacatccgagccaaaggagataaaatagcaatatggaccacaaactgtgaaaacaaagaggccatcacacagattggtcgagtttataaggagaggttaggcgttccacccaaagtgatcatcgggtaccagtcccacgcagacacggctacaaagagcagctccacaaccaagaacaagtttgttgcttaaaggttttgggtttgtcttctttccttgttttgtttttttcaaagtaactgaaagtttattcaccaaaagccttttctaaaatggaacaagtcttgcctttactttgtcagaacatttgtttgtggtgagaatacaataaaaaaatagtaggtgtaaaatcctaatgattgacttcatcatgggatagaaatgtaagcagtttattggaggtgattcatttgtgttccaacaattctctcagtttttacctttttaagttgtttatggagaattgcttaataaaggtacatgaatt

SEQ ID NO: 3 - DNA, 2862 bp, Oryzias latipes
  misc_feature: cDNA for gene 3
gctgcttcatcttcttggccttggtgtaaaagtcaaagccaagttctggtggttgagttctttccagttcctttttccaatgccttcacactgcaacatgcggaagcctgagaaaatgtttttgttattgaacctgatggctgcagtttctgtcttgagtcaagcatggccaaatgtgaagcatggtctgcagtcaaccaatggagttagatcggactgtgcaggtaatctgatgaggctttcattggatgaggctcttgcagttgggaatcaacttcaagtggaagcaatcaatggcacacaacgtattttggtgacacccagtctggctgcccagtgtggatacagcatggagtctgacccatggggaaacaccaggatctacatgtctctgttgggttgctttatggttagcaaggatgagtccaccttcagcaccagcttgaaactgcacatgtacaagcacagtccctctgatgtggtcagccatgatgtgactcagacctgcagctattctcgctgggccttcagagatgtcctctgtgacaggaactacatggaagtgtcagctcacatcgctcccagtcaacaaacaaaaggacaagttcagaacaaggacaactctcaaacaaataagcttcctggtgactccaatgacgcccctggaatctggaagatgaccttttacactcctgaacctgttgcgatggtcctgaaagaagccgaacaagctggttatggtgcaacaatgagacaaactcgcctggcaatcagaagcccctatcatacttcagagacttattctgaagatgttgctggagtccccatggaaatcctcaaagtgagcgtttaccacaaaactcaagagggactgaatgttgtcaacttggcagctgcttgccccacaggtggcattctcttcactgacgagttcatctcctggcacatccctcgccgcgtaactcctctaactgagggcaaggtcaagattgtagagctgtacatgggaatcaatggacagaggctggaaaaggctcaaatagctgcaagaggttactccctctcaaccacagagttccacattgttgttgacatcccagtgggatcacctgacggctactacaagagccatgctccagacttcctgtaccacatcacctacaccattgagccaatgcttgaagttctatggaggccagagaatccccaagaagaaacaaaatacaaaattctgtttcccatcacaactccaccaatccttcatccaatcaacacctttgaccgcacaatcccagaagagagggtgtttaatgtccatgtgggggctttccttcatgatgtggtgctgaagaacatcacattttctactggagtccttactgttgaagagtgcaatgccaaagggtttgcagtccaggaacatcttctctccaacggcacaaaagtgttctctattcaagtggcctttgacaccgatcccatcctgaagcataatcctgagcccttggttacaacctacttcctccctctgatctttggttttgctgttctgcctgaggatctcccttttgcccacaaagtagagttgcaggcatctctgcaggatgttgttctgccaacactcactggaacctgtgaccaggagcacttcttcgtttccgtcaagtacggcagtcaagggcatcacttcaagaccatggtcggccatcaggacctgacacctgaactgggagagacactagcctatcaggacaatggcacacacttcagcattgtggtgccttatgctgcccccgtcactgcatttgagctgatcacaacaaactcagtcagaaccaggctgaatatgctgctgtgggactcaatcaatcaatgggttcttggagacctttacttgtcttgcagtttccctctggcaacaacaaggtgctactcaaatggaacaatcagtgccatcgctgtcaaagtagagtctgtgccaaacctcagcccaagctggctgaccttgaaggaccagtcatgcacaccagcattcattgatgaccgctttgctcactttgtcttccatgctgactcgtgtggaaccactagactgttcttcgacaactacatgatgtacgaaaatgaaatcaggctgaacttcaacaggaaaggagttgcttacacatcaccagttgatcctgattacaagcaaaccatctcctgctactatgtggtcaatgatacccagagaatctcctttgcttctcaaccaagactccatgaacccaaagcagagattggattcggacacctggtggttcaaatgagactagctcaggatgcttcatatcaaatcttctaccaaactgaagactatccagttcagaagttcctgagggagcctctctactttgaagtggagctgatgcagtcaagagaccctaaactggaactggtgctggaaaactgttgggcaacaagcaaagaagagaggaactctctcccaagctgggacatcattatcaatggctgtgaaaacccagatgacagttatgctgcagtatttcaccctgtaatgaaggacagcagagtttcaatcccatctcatgtcaagcgcttctccatcatgatgttcgccttcatccagaatgaccaagttctgaaggatgagattcatgtccactgcaatgtagtgatctgtgatgccaacacacctgcagaaggcatctgcaaaggccaatgtggttactcatctggcattaaggcacaccaaagcacaaaaaagggacaaagaccacaaagctcaaccaaccacaagcagatctcctctgggcgtattctgttgaataactgaatgtccaaataaaaatttcttaaatg

SEQ ID NO: 4 - DNA, 1278 bp, Oryzias latipes
  misc_feature: cDNA for gene 4
agtttgtagtcactaacaccataatggggtttggcaagtggttgtttggtgttgtgattttggcttgtggtgcgtcagcacaaaaccactggacaccacagaaacaccagcccctgtttcctcatcaagcgcagccactggtcactacttttgataagtgcaatgtggaggagggtgacaagattgaatgtgggacttcggatatcactgtggagcagtgtgaaaaaatcaactgctgctttgatgggtggaagtgtcactatggaaaaggagtgactgtgcagtgtaccagggatggtcagtttgtggtggttgttgccaaagacaccactgtgcctcccattgatgtgaactcgatcagtctgctggagtccaatggtgacttctgtggtccagttgacagcacctcagcctttgccatttttcagtttcctgtgactgcatgtggtactacactcaaggacgatgaaaactacattgtctatgaaaaccacatgtcttcatcatatgaagtaggagttggacccagaggatcaatcacaagagacagtcattttgagttgttgttcatgtgcaagtactctggctcagctgtggaagctcttatcttggaggtcaaccctgttcctgctcctcaaccagttgcagctctgggacccctgagagtggagctcagactggcaaatggacaatgtctcgcaaagggttgcgtagaagaagaggaagcatacaactcattctacaacccagctgaatatccggtaaccaaagtgttgaggcaaccagtttatgttgaggtcagggtgcttggaaggtctgacccaaacattgtcctaaaccttgatcactgttgggccactgcaaccccaaatccccaaagtgttccccagtgggacctccttgttgatgggtgcccttaccaggatgacagatactcaacgatattggttccagtggatagctcttctggccttgagtacccaactcattacaaacggttcatcagcaagatgtttgcatttgtggttccagaaatatatactccccaggagacggtgtacatccactgtgccacagttgtgtgctacccaagtagcacaaactcctgtgaacaacgctgtcatcgtcagcgaagagctgcggtaaagattccttcaagtcagaaggctctggtctccagtggtaaagtgatcctgatcaagagtcctgcatctcacttgaaaagttaaaattgctcttggccatctgttttaataaagcttgtcaaagctcatgctc

SEQ ID NO: 5 - DNA, 1003 bp, Oryzias latipes
  misc_feature: cDNA for gene 5
ctgggatttggcaatgtggttctgtttgggggctgttatggcagtgaatgcagagctgaggacggactgcaggcctgactacatgtcactagtttggactgacagccggttgcaggctgatccttccctgtttcgtcttggtagctgctttcctgcgactatcagcccccgggaggtggcttttagcgtgacatatgatgactgtaacttcaggagacttgtaactggaaatgagcttaactacaccaatgacctcatctacacatcttctcctgactcgtatgtcaacccttttagtctcccggttgtctgctcatttgagaggcccaaggactggtatcctatgacttatgacccagagttttccacatatggtgtagaagacttggtgtttcaagttggactaatgaatgctgacttcacaggaccctctgagtcaaatgtgtaccccctgggctctatgatttctgtcatggctgctgtggaccagcaagaccatcagcctctattgctgtttcttgaagagtgtatagcctccaccacacctgaccttcaccctggagccgacttgtatccaataatcacaaataaaggatgcctggtggatagtaaagtttcgcgttcaaagtttgaaccaagggagaaactgtctgagattcacttgtctgtgcaagcctttaaatttgctttgggacatgaggtgttcatccactgcaccctggttgcatgggatccaaatggactagatgacaccaagaaggcctgccactacaacaaagatcatggctgggagttgctggacaaccctgcatataatggcctttgtgactgctgtgaatccacctgcaagtcaagaaaaagaaggaatctgtctgaaaagcatggcttggagaaaaaggcagttgttggaccactcacaataacggcctaaagtcctgaaccagcttgtgatttattttaagctgtaaatccttgaaatgacattaaataaaatttgaaaatg

SEQ ID NO: 6 - DNA, 1110 bp, Oryzias latipes
  misc_feature: cDNA for gene 6
tggggacttgcatttaccttggagctgtgatccttgctgtttttattgcagctggtgctgagccagatattaaagttgtctgtgcaagagactctgtgaaagttaaatggagggtttcttctccctttgtgccgtatgctgctcgtcttttccttggaagttgcatgccatccaagtggcaacttctaccctctggtgatggggaggcattttttgattacaagttctctgaatgcaaatttactaaacagataaaaggaaaaaatgtagtttataggaatgaactcagcttcaggccacaagcaaagggaaggcccgttgtttttaagcagccaattgaatgtgtctataagaggcctgagggttggattcctccattcttgaatcctggatctggtgtgtctgagggtcacagtaatctggtttttcacatggcgctgctcaatgaacaactaaccggtgtggcaaagacaaatgtgatccccttgggttcattcatgcctatatgggcagcagtggagcagaagtcccatcagccacttctgctgctcatggatgaatgtgttgcagccaccacaccaaaactggatcctggcagccaggttcaccaaattgttggaaatcacggttgtctccttgaaagcaaacgagggagtgcagtgttccttccacggtaccactcatctgccattatcctttacattcaggccttcaactttggacttggtgatgaggtctacatccactgtaatctggttgtatgggatgagggtgctcttggccagagtagaaaagcttgccatgtaaaagaccatggaagttgggaactgcttgatgatccatcacgaagctctgtctgtagctgctgtgactcagtctgcagttcaaaggccaaaagatcagtcggcgagtctggtacatcaggctacaacacagtgttgggaccattggtgataaaagacctgtctgctgcatctaatgctacactagttggatctctccctggaagaagtctgtagcggctgaacacgtgaaggaaaagtctctgtcggagtgaaatttcagctgtagtctgtatgctttaatccaaataaagtttctttaaatctt

SEQ ID NO: 7 - DNA, 1258 bp, Oryzias latipes
  misc_feature: cDNA for gene 7
cagttttcttttaaaccatattttatccttacttccatactggctttcttgtggtgtccctaacacctcctaaggctcgtcatgaagaagtttttggctcattcagcctccctcgtgctgctcatttcttttctccgtggagttgcaattgccatccgaactctaaaagaaggtcctatgattgatgcagacggaagggaatataaaactgcttctttgacagaagactccagcccgagagcaagcgatagtgtccgtgtggagtgcacagaagtgtccatgattgtctacatacaagcagacttctacagaactggacgccttgtgtctccaggggacttgtttttgggaggtgcagagcataagcaggacagtcggtgtcgggctgttgtttctggacacaatgagtatgtcattgaagctggcttgcaagactgtggctccaagttgactataacggatgatgatgtgatctactcaaacaagctggttttctcaccagtttccaatcaccacactattacaaggatgactgatgctatagtccctgtgtcctgccattacaaaagaacacacactgtaagcagcagtaatactgaacaggcgcccatgacgttctctcagtcagcaaagttctcaaccaagaactctgctttctctttgaagctgatggctgatgactggttaacggagatgttgtccagcaagtttcatcttggagactttctgcgctttgaggcaaagtatacgggcccagagcccaggcagctttttgtcgaaagctgcgttgccactctgacacctgacgcaacgtctgtacccagatactacttcattgaaaaccacgggtgcttcactgattcaaaagcggggtcagttgcctcattcctacccagatcgagagccaatttgcttcagttccagattgatgcctttctgtttcgtaatgatttgagaaacactatctacatcacttgtaacctgaaggctaccctccaaatgaggaccaccttgactgacaaggcctgcaattatgtgcattcaagctggaaaagtgtggatgagaacgatagtgtttgttggtgttgtgacagtatttgctaccaaagtcttcccagagatgatgatctttgtgacattgtcactcttggtccactgaggattatctctaacaagtaaagagcaagtgacaaactgtctgccagaggctcttggattaaacctgcgcaacggtcttgtatgtccaaaaataaagttt

SEQ ID NO: 8 - DNA, 1509 bp, Oryzias latipes
  misc_feature: cDNA for gene 8
gcctcgtcgctccttccaaactctgaggatggactgcaacctgcggctggccgtttcttgctggatcatggtcttttcttgggtttcccctcttacagagagtcgccagacgaacagccgaggttctacagaaaggcacttccaacctccagtcgggacgcacggcggtctgcagcctcggctctactcggtgaagcagcagccggccccggaggtacccgaacagcaccgccccgtcacggtcatatgccacccggattccatggaggttgtggtgaaggccgacatgtttgaaacgggcctgaatgtggacggtggacatctgcgactgggttccaacactctgggcgcgggcggtgagtgcggggcggtccagaaaggagaggacgaattcaccatctgggccctgttgtccgactgcggaaccaaactctcatcaacagaagagaagatcatttattccaacgttctgatctactcacccgaaccttctgctgatgggttgttaagattggaagctgcaactattccagttgaatgtcattatgacaggagatactctgttgatggcatttcccttgaatcaacttgggttccctctgtctccacaacttctgtgaacgaccagatagatttcaatctgaaactcatgactggtgactggcagtctgagagggagtcttacacatatttcctggctgatcccattaattttgaagtctctgccatagtggaaaatcacgtccctctgcgggtgtatgtggaccactgtgttgctacggcaactcctgatgcagaggctaatttaagatatgaatttattgaacataagggctgcctcgttgatgcttaccttacaaactccggcgcacgtttcctaccaagaactgaggaacataaactgaggtttcagctggaagccttcaggttctatcaagaacccagcaaccagatctacattacttgtgctgtgaaggctgttcctgctgtacaggccgtcagttctcagaaccgagcctgctcctttattgagaacagatggcaatccatagacggtggtgatcaggtgtgcagaagctgtgacgtgttcaggcggggtcaggaaccgcaagctgtgccatctcctaaaatgccagtgaacgccaaagaccaaatcggtctttcacagaaaaatatagtccacaataaagccgagcatcaaccggcttcttacgtccatttttggccgggagcttatcagagccatcactccaaacctcagcagtccaacagatttatgaagagggatgctgacaacaaatttcatcaagctgtccaactggggcccctcgttgtgctaccgtcaagaaaagtagtttcagtggcaacaaatttttctacatggtcagaaaagaacaacacctcctgagacctggaacacctgatggaggagtctgagttcgtttgagagatttggtcttgttgggctaaagaattttgctgcaatccaataaattaagtttctaatgt

SEQ ID NO: 9 - DNA, 1929 bp, Oryzias latipes
  misc_feature: cDNA for gene 9
cgcggccgctgggtcagcgaggagcacttgcagtctgatttttgggtgctgctgcttcagtcattttgatggctccctcacggttaaagattagctgcctttttgggcatttgactgccttctgtcttcagttgacacttgcgtttccaccattgctttacgtgccgcctgtttctctcaaaagccaaagctccttgccgagtgtggtccagcagccagaggagccggtgcccgttaacacagttggtgtgctctgccaccctgattccatggagctcagcatcaatgctgacctgtttgaagtgggagcgcctgttgacgtccgtgagctgcgtctcggagtcgagcacagcgactactgcagtgcaacagcatcatcagactctgagtacagaatcctcgtgggcctggaggactgtggcaccaaacactggatgacagaagactctctggtctacacaaacctcctgatttatactccccttcctgcacttaatggaataactcgaatggaggaggccgttattccaattgaatgccaatataaaaggaagtacagtttatccagctcatcactcgtgccaacttgggttcccttcacgtccacacaagctgctgtggaaactctccagttcaacctgaggctcatgaccaatgactgtgccataactgtaaagaaacctcagaggccaaagaaatcctggcagaaacgtctgaatggacacaaccagccctttgtggaggaaaacctgcagcggctcttcgctctgcgcatgcgaatctccagacgtgccaaagtggaaaccaaccttgccggtctcttcaacgagcgcaagatccctcatttcgttgacccagaggtcaacctgcgtaacctgtttggcatcaaacctcccattcctttggaaagtcctgctgtggcaccagttaaatgaaaattcagatctgatgagttattttaggaagttgggtctttttagtttaaattgggcatttatgttcaaagaatgttaaaatttggctgcacgaaaggggtgccaacacgttcttcctcggcgagccaatcaacattgaggcatcagtcagggtggatcatcacatggggctccggctgttcctgaacagctgcgtggccacacttgaacccaacatcaaatctgatcccaaatatgtctttattgagaatgggtgcctgctggactcccagcttccaggctcaaagggtcacttcctgccaagaacaaaagacaatatactgcagatgactatcgattcttttaaattccataatgacgaacgaggacagctttacatcacatgtcatctcaatgcggtgccagtggatgatgcagaagcacaaagcaaggcctgcacatatgtaaatggaaggtggcggtcagctgatggaaacgactacctgtgtggattctgtcaaagccccaatgaagccagtaaagcactcagcatccctggagtctttaatcctcgtagctttggaaaatctgcagacatggagcccatgtggagaagtggactcaagactaataaggtttgggaacatgaagccagactggggccagtgactattctgccttcatggaagagtggggctcttgctgcacatgaactacctccagttctccataagatccacaaaacggcgctgtacggcagccactggaggagcggcctaaacacaatcgagaagagcctggttccagaaccatcaagttcagaactagaagaggatgacagtgatgactatgatgagtctgactctgacctggcgtttgtgatgaagtccttggagattgcccataccaacactaccttccccatcgtcacatatgacctggagccctctaagatgaatgcaactgctgctaaccctgacctttctgataaacatgacccaaagaaataaaatgc

SEQ ID NO: 10 - DNA, 919 bp, Oryzias latipes
  misc_feature: cDNA for gene 10
gaagattagatgatgaaagctgagaaagctgttggggctgggccccagcaggtgttcacctgcacccacgccggatgtggagcctgcttcccgagggagtggaaactaaaggcgcacgaaactgtgcacaccggagagcgtccttgcgcgtgcccaacagccgggtgtggtagtctcttcaagagaacatcccatctgaaaagacacgtgctccagcataaaggagtcaaagggttccaatgcaagtttgcaaattgcgcaaagagtttcatcgacgctcaaaggctgaagaagcaccagaacagcgctcatgggaatcacaaattcaagtgtaatcaacccaagtgctccttgagcttcaagaagcgcagattgttgaagctgcatttaaaggagcataatgtccatccgaacttcaaatgttctaacattaggtgtactgcaacgtttgactcccatattgcacgcaaagcccatgagaagaagcatgcaggttatagttgccctcacaaagactgccaggttgttgaacacacctggagcaaacttcagaggcacctggccaaacacccagtctcatttacctgtggcgtgtgcgagaaggtgtacgacaaagcaggtgctctgcggcggcacaaacggatccacgcttcccataaacctgtgctgctgtgtccaagagctaactgccaggcctacttcaccacgacctttaacctagagcatcacattcgcaaagtgcatctccagctcctgaagtataaatgtttcttccccgactgcccacgcacatttgttatgcgggagagcatgcatcgacacatggttcatcatgatccaaaatttcctttaaagtaaaaggctaaataaaaatgctctgtagtgttattgacccaataaaatgagttttgttttgctc

SEQ ID NO: 11 - DNA, 1557 bp, Oryzias latipes
  misc_feature: cDNA for gene 11
ggaaagcttcagttggtaagacgtcagtggctcacattcacctacaaccatggcgaaggagaaggttcacgtcaacgtggtcgtcatcggtcacgttgacagcggtaaatccaccaccactggacacctggtctacaagtgtggaggcattgatcccagaaagctggagaaatttgagaaggctgcagctcagttgggaaagagttccttcaagtttgcctgggtgctggataagctcaaagctgagagggagcgagggatcaccattgatatctcacttttgaaatttaacactcaaaagtacaccatgactataattgatgctccagggcacagggacttcatcaagaacatgataactgggacgtcgcaggcagatgtggccctcctgatggtctcggcagccaaaggggagtatgaagccggcgtttccaggagcggtcagaccagagagcacgccctgctggcctacacgctgggggttaagcaaatcatcgtctgcgtgaacaagatggatctgaccgagcccccttacagccagaaacgctatgaggaagtgatgcgcggcgtgagcggcttcctgaggaagatcggctacgacacgaatgccgtgcccttcgttccagtttctggatggactggggagaacatgatcagcgtaacccagaagatgccctggtaccaaggctggaaaatcaggcggagggaagggccttcaactgggaagactcttcttgaagttctggactctattcagcctccggtgcgaacaatcaacaaacctctacggctacctctgcaagacgtctacaaaattggaggagttgggactgtgccagtggggaagattgaaacaggcgtcctcaaacccggcatgaccttggtgttctctccagctaagctcaccgcagaggtcaagtcaattgagatgcaccaccagggcctgcagacggctctgccggggcacaacgtcgggttcaacatcaaaaacgtgtcagtcaagaacctgcgccgtggggacgtggccggcaacgcccaacaggatccaccttcagatgtccgcagctttgaagcacaggtgattatcctgaaccatcctgggaagatcaaggcgggatactctcctgtccttgactgccacacgacacatgtcacatgccgcttcaccgagctgaaggagaagctggaccggcgcaccggcaagaagctggaggagcagccacaaaccctggtgtctggagatgctgccactgtcaaactggttcccgtgaagcccatgtgtgtggagagcttcttcacataccctcctttaggccgctttgcggcaagagatctgaagcagacagttgctgtaggagtcatcaagtcggtggagaaagaccaggggtctaaagctcaaaagcttcaagtttgtaaataagtgctctgtaaatgtatgctgcgaaaaagctttttattttccagtgaatttcagtttgtttaaaatgtataatttgaccagaaaagatgcagctggtatatttctgttaatttaaacaaataaaaactggtttggag

SEQ ID NO: 12 - DNA, 865 bp, Oryzias latipes
  misc_feature: cDNA for gene 12
tcattgttcacagttgacgggctgctttaagatggcaaagaaggtgctgatcgtgtatgcccaccagagctctagttcattcaattctgcagcaaaaactactgctgtggaagttttgaccactctgggctgctctgtggaagtttctgacctgtatgctatgaattttaaagcaactgccactgctgaggacattaaaggtgaccttaaggatgctgaaaattttagctacttggatgaaagtaagctggcatgggaggaagggagactaatggatgacatcactaaggagcagtctaaggtcattgaggccgacttcatcatctttcagtttccaatgtactggttcagtgttcctgccatcatgaagggctggattgaccgtgtgctcacaaacggctttgccttcacacaagagaaacgttacagccagggaatcttcaaggaaaagagagccatgctgtccttcaccactgggtcacttgaatcaatgttcagtgctactggcattaatggagacatgaatgtcacgctgtggccgctgcagaatggaatcctgcactactgtggcttccaggttctggcccctcaaatcttctgggctccattctcagcaacccctgaagctcgtagctgcatgttggagggctggcgtgcacgactgcaaggccttctagaggagcagcctctgtcattcatttccctggactgctttgacaaaaaggggttccaattgaaatctgatgtccaggagaagcacgcagccaaggactttggtctggcggtgggaatccacatgggaaagcctctgccacctcacaaccagatgaaagctggatcttaaacatgtttgcttgaatgatga

SEQ ID NO: 13 - DNA, 705 bp, Oryzias latipes
  misc_feature: cDNA for gene 13
cttttgggttctagtttagtggtaactgtagcagaatgaggtttctgtggatttcttgtcttctcattggaagcatctcctgtcttcctcaaggaggatatgatccatccctgtttctatacactggacaagctccatcctatgagaaaccttctgcacagtccagtggctacagcagtccacaaggctattacagtgctggcaccaacactgctggaggctctacagacagtactgctcccatgtggtactctgcttcctatcctgaacaagagccagccaagccaacttatcagagacctgcacagtccagtggttatggcagctactctggttctggatctcaacagtctggttcccagggcgctcagtctggagttccaggcagccagcaccaggttgaacaggagagctggagctcctcatctgacgacgaggaagagcccgagttcactccagtgagtgaggaggatcaagtgtacgctttcaagtctcgctctcgctacaaccagaaacggctgctgttcagtcagttccgctacaccccaacagaaccccgtgttcctcaagaaccagtgttcccgtaccccggcaagtctcatcagggcaaaggttcagctaaaggacgccgctaagatctggcttttgttttgaggaaaccgactgatttattctgaagaataaattaaaatcttaaaatgttac

SEQ ID NO: 14 - DNA, 725 bp, Oryzias latipes
  misc_feature: cDNA for gene 14
ctgaggtttctgttctttgggggtggagcagctgcagaaaaatgaggtttctgtggatttcttgtctgctcattggaagcatctcctgtcttcctcaaggaggatacgatccatccatgtttctatacactggacaagctccatcctatgagaaaccttctgcacagtccagtggctacagcagtccacaaggctattacagtgctggcaccaacactgctggaggctctacggacaatattgctcccatgtggtactctgcttcctatcctgaacaagagccagccaagccaacttatcagagacctgcacagtccagtggttatggcagctactctcaacaatctggttcccagggtgctcagtctggagttccaggcagccagcaccaggttgaacaggagagctggagctcctcatctgacgacgaggaagagccagagttcactccagtgagtgaggaggatcaagtgtacgctttcaagtctcgctctcgctacaaccagaaacggctgctgttcagtcagttccgctacaccccaacagaactgcgtgttcctcaagaagcagtgttcccataccccagcaagtctcatcagggcaaaggttcagccaaaggaagccgctaaagatctgactccatgtgttctgtggctgttggcaaatacctaaatgcaagtgctgtgtggtctttaaaataaatatttaaagttcgactgtcgtgg

SEQ ID NO: 15 - DNA, 728 bp, Oryzias latipes
  misc_feature: cDNA for gene 15
ctgtctcttgtgtataatccagcagcattcacatcatgatgaggctcttgttactttcatgtttcctccttggacgtatcacctgctatcctcaacaaggtagtggtggtcattttctcccataccaaggtcaggctccatcctatgaaaaaccattgatgcagtctggctacagtggctttcctggtgtatacagcagcagcatgaacacagctggaggcagtggaagtcctcccatgtggtactctgcttcctatcctgaacaagagccagccaagccaacttatcagagaccagcacagtccagtggttatggcagctacggcagtgttgacagcagctactctggttctggatctcaacagtctggttcccagggtgctcagtctggagctccaggcagccagcaccaggttgaacaggagagctggagctcctcatctgacgacgaggaggagccagagttcactccagtgagcgaggaggatcaagtgtacgcttccaagactcgctctcgctacaaccagaaacggctgctgttcagtcagttccgctacaccccaacagaaccccgtgttcctcaagaaccagtgttcccgtaccccagcaagtctcatcagggcaaaggttcagccaaaggaagccgctaagatttgtgagggccactgaagatcacctgatttgacttttgtaatctacagactccgctaataaatgaattaaaattc

SEQ ID NO: 16 - DNA, 729 bp, Oryzias latipes
  misc_feature: cDNA for gene 16
ggcacgagataatccagcagcattcacatcatgatgaggctcttgttactttcatgtttcctccttggaagtatcacctgctatcctcaacaaggtagtggtggtcattttctcccataccaaggtcaggctccatcctatgaaaaaccattgatgcagtctggctacagtggctttcctggtgtttacagcagcagcatgaacacagctggaggcagtggaagtcctcctatgtggtactctgcttcctatcctgaacaagagccagccaagccaacttatcagagaccagcacagtccagtggttatggcagctacagtggtgttgacagcagctactctggttctggatctcaacagtctggttcccagggggctcagtctggagctccaggcagccagcaccaggttgaacaggagagctggcgctcctcatctgacgacgaggaagagccagagttcactccagtgagcgaggaggatcaagtgtacgcttccaagtctcgctctcgctacaaccagaaacgactgctgttcagtcagttccgctacaccccaacagaaccccgtgttcctcaagaaccagtgttcccataccccagcaagtctcatcagggcaaagtttcagccaaaggaagacgctaagatctgtgagggccactgaagatcacctgatttgacttttgtcacctactgactctgagctaataaatgaattaaaattccctc

SEQ ID NO: 17 - DNA, 696 bp, Oryzias latipes
  misc_feature: cDNA for gene 17
ggcacgagagggtgctgttggtttgtctgcttgttggaactgttacctgtgttcctcaaggaggtggagcttatcagcccagagggcatcctccattctctggacaacttcagtctgagccagtctatgaaaggccttccggacagtcgggttatagtggcgctccgggatattttaccagtggaacctacactacaggaggcagtggaagtcctcccatgtggtactctgcttcccatcctgaacaagagccagccaagccaacttatcagagaccagcacagtccagtggttatggcagctacggcagtgttgacagcagctactctggttctggatctcaacagtctggttcccagggggctcagtctggagctccaggcagccagcaccaggttgaacaggagagctggagctcctcatctaacgacgaggacgagccagagttcactccagtgagcgaggaggatcaagtgtacgcttccaagactcgctctcgctacaaccagaaacggctgctgttcagtcagttccgctacaccccaacagaaccccgtgttcctcaagaaccagtgttcccataccccagcaagtctcatcagggcaaaggttcagccaaaggaagccgctaggatctcgttgagcgtcactcatgatgttttgatgttgagctgctgaaaagaataaaaaaaatac

SEQ ID NO: 18 - DNA, 731 bp, Oryzias latipes
  misc_feature: cDNA for gene 18
cttgggcatcagtcaatagcaaccagcagaatgagggtgttgtggatttgtctcctgatgattggaagcatcaactgccttccccaaggaagtgtcccaaatatggcagtccctcgctccatgtggcttcctccttactatgggcaagaaccatctagaccatcctatgaagagccttctggacagtatggtggttatcccaccttcccaggatcttacagcccggagcctcaaactgggggcagtggaagtcctcccatgtggtactctgcttcctatcctgaacaagagccagccaagccaacttatcagagaccagcacagtccagtggtcacagcagctacggtggtgttgacagcagctactctggttctggatctcaacactctggttcccagggcgctcagtctggagctccaggcagccagcaccaggttgaacaggagagctggagctcctcatctgacgacgaggacgagccagagttcactccagtgagcgaggaggatcaagtgtacgcttccaagactcgctctcgctacaaccagaaacggctgctgttcagtcagttccgctacaccccaacagaaccccgtgttcctcaagaaccagtgttcccataccccagcaagtcacatcagggcaaaggttcagccaaaggaagccgctaagatctgtggtctcctgggttactgatattttcaaatgtgaaattaaagtttcctctgac

SEQ ID NO: 19 - DNA, 761 bp, Oryzias latipes
  misc_feature: cDNA for gene 19
tgttttgttttgggtattagtcagtattatccagcagaatgagcagggtgttgtggatttgtctcctgatgattggaagcatcaactgccttccccaaggaggtgtcccaaatatggcagtccctcgctccatgtggcttcctccttactatgggcaagaaccatctagaccatcctatgaagagccttctggacagtacggtggttatcccaccttcccaggatcttacagcccggagcctcaaactgggggcagtggaagtcctcccatgtggtactctgcttcctatcctgaacaagagccagccaagccaacttatcagagaccagcacagtccagtggtcacggcagctatggtggtgttgacagcagctactctggttctggatctcaacactctggttcccagggcgctcagtctggagctccaggcagccagcaccaggttgaacaggagagctggagctcctcatctgacgacgaggacgagccagagttcactccagtgagcgaggaggatcaagtgtacgctttcaagactcgctctcgctacaaccagaaacggctgctgttcagtcagttccgctacaccccaacagaaccccgtgttcctcaagaaccagtgttcccgtaccccagcaagtctcatcagggcaaaagttcagccaagggcagccgctaggatctgtcatttcaggatcattaatccatgactgctgtgcaggttttcatgtaccagtctaataaaataccattcctg

SEQ ID NO: 20 - DNA, 629 bp, Oryzias latipes
  misc_feature: cDNA for gene 20
tacgactgcagtgaaacttttctagtttaatttagcaggttgctgcttttcaaagaccaacgtgatgcttcagtctgttatgaggttgttctatatcagccttttcctcttatactttggagcctgtgttccacttaaaaaaagtgaaacttctctcggctctggcttcagttattccagtcctgggtttggctctgactactcgggacgcggttcttccatttttggttatgactctcgtggcgattttggttccggctcgccgagaaagcaggctgcaggctttgatcgtttcattgcagaaatcttgagcctgagaccttctcgttctttcccacgccgtgcctggacctccaaccaggtgcctctcggcatggttgagccacgccccgtgtacccttcatcccacgttgtcagaacgagcaatggctaccagcgagctcgggacttccggagtgatgccaagtacgctcaagatatttttgaccacatagatgaggacggtcaacaagagggccaccaaccgactggaccaacgggtcaaaagacctactgaggtaaaggtgaagaatctactacctggacagacagacatacatacaaacaaacaaataaatgttaatctgtct

SEQ ID NO: 21 - DNA, 843 bp, Oryzias latipes
  misc_feature: cDNA for gene 21
gtttaaggagggcagtgaattcccaaagcttgtgttggagctgcaggcaggagccatggctgctgggttcctgattagcaggtttctgctgatttttgtgttgagtgaactaaagtactcatcggtttttccatcagttgggagttgggggagttttcaggtaacgttcgaccctggctttcaccatcaccacaaacccactaatccattgatgaattactggttaaaacttaaggagttgccaaatctttggcaccacacacggaacaaaccgctgtgttgtgatggtgactcaaaggtgcctcgccatcctgtcaagcctccagtcccaatctgtgaaccaacaaaccagggaccagactgtcctgtgaaacacggcccaacccatcctgaaccaaagtggcccatcgtcagtcatggtccatcccatcaccacttgcactggcctttctttcgtgtcctgcatggacttctctgtcaccagcacccttgtcctcatgcccacagccacgcctacgatgatgaccgctgttctgcacatcagcatcctcgccactgtggaaaacacaaacaccaccatgggcctgtattctaccaccctcaccatgggcctggacatcatcatcatcatcatcaccacaaccaccagcagcaggtcaaggtccccccgcatggctgtgaacctcacagtaaattatgttcatgatgtccaagaagtgattcaagctttgttggctcctctgagtcaaagcagcctgacctccacggaggactctacttccaggttgggccaggaatactgagctgtgcaggtgtctgcatcaataaagatttctgatagag

SEQ ID NO: 22 - DNA, 13502 bp, Oryzias latipes
  misc_feature: Genomic DNA for gene 1
gatcttaaacccgagcccgagtcagaacccgacccgacgtggggcactaaatgacaatcattacgttcgggtcggttcgggccgggcttctctctcgttgacttttttaataaatatattgtaattgctgcaaagaagctccttaggcgcgcgttttcaaacaactatttattaatcctgtatgtcagaacggttaccgcacgaaccgaaaggcacagccagcttcttcttactcagagggagaacgttcaccgaacgcacacacacacacacacaggtagaccccgccccctctatcccaccagtcaccagcccgccgttgaatgacacacactgtggtccagcaactggcagaaagaggggggcgccggccacccgcagctctgcgcacgtgtcgtgaatgtaatcttccctccgcaagcccaaatctatttttttaaggtatccccgatatggagcagcaagaagtcaaggataaacttaagacaggggaactgaaaggcaaacacgcaccgcggctgcagaatttctccgcgtaatgaactttcactctctttgtctggcctctaattcgagtaagccctggctgcagaattgctcaacatagtggaatgctggaacgctgaggaatgtgaacacgctgcattccactatgttgtgcaattctgcaggctgcagccaggggtagttgtcttattctgtgttccagcgttatttcgttgtgcaaatgcatttatcatgtagggaggaatctatgcgcagcgcttccggcggaactctatgccacaacagcgagcttgactacgcgcgcggctgcagcgaccactctctgttcgaaagaaaataactttcatgatcataaaaatatgtagattatttaacctttaagaaatctggcgtttttttctgccaaaaatactatgggataaattaaactttcgggtttgcttcgagctcgggcctgcaaatcaagttaattggtcgggttcggaccgggttcggctttaatgcccgtgggcctgtctcgggtcgggctggattctttcggtccgatcttacctctaagctgaactcacagtctttggttcagagggttaactgcaccacaggcaaagcagcaatgaggagagtggatgcagatttaatccaaaaaagagaaaagacaaagtaagtttttaattaaataaggaacagaaactaaaattcataacagaagctgactagtacctaaacatcctcatgaagaatgttttgatttattttggtaatgaaattgcttttgtaactgaagttgctgtttaagtgaacttaaaaaaccctggttatcctatctgcttatacatttctaatgctgttaatttattcctttctttcatctattgttatcacactagttttcataattacttgaaacctttgtacttggctttgctaaaaaaaaaaaaaaaacatttcagggaattattcttttttgaatcttcaatacttcaatacttcaatgtttcaagcagtagcatttgtttttctttcttgttgcatcaagtggcctcttggcaaaaaagtcaattttataccaaccaaatgtcaatgcagggcaatgagctctgaaatctgagttttagattgaagcaaaacaagaaatatgttccagtttagaaacctccaaggcaacaaactcaagtgtcaaccacaaagaaaaagtataaggcagaaaaaaactgttctataaatgtcttgaagtggcaaatttgccacaacagcccccattattctaatttatttgtgtcctgacattcatggaaacaggttgtttacttcagcataaggattactgcacagaaattgatgatacattcacacaacataaagtttgtgtcgaatggcttttgtgtaaaggggttgtttttttcttgtgtttctttcaagtgggccggtaaaacataatgtaattgcatactgtttagataatcaagtgtaattatgccaggcatcctgacgttcttttgaggagttcaactatacctggaaattatggcgggagaaaatgttctttgtatatgtgtccctgacctaaactggagtgttctgcaacacaatctcaatcaaatcaagagatgaggagaaagtttgctgctagtctaaatctgcaacatatacacagtacatctgacatactcaagctagccagtttttcaaagtccatcaaaatgtttaaatgtgaccctgcgtcattttcctgattggtatgattcagggttttgagaagtctcaaatttaaaaaatctttagtaaaatgcagtttatttgtgtttctctttgtgcctctgaaagattcacatgtacttaagtcacaggaaatggtgctgcaaaaataagagtatttccccagcctggtcacacttctgtgggatggcatcctgctcttcaactacccttagcccactaggcacggacatatctattggatatctaaattatggctattttaaagctcacagctatagatatgaattatgcatctgacagacatcaattcatagaggtctattgcatatccgggtttagatctctagcaaacgactttcccgcagctaatcgtgtcacggtcccatcatgcttgtgcattgtaaacaagccgtttgggtaccatagtttcttcccactggacacagaatgtctagacatctattgtagatgtctactggacatctcagacaactctattttgttacactcagatgcctactaatattgttctgcatacagaggtctcttatctacctgtagacatcaaaccttggatattttttagatgtctattacagaacttttttttttaatttatttactggatataaaattgaaaacccagactgaacatgtttctatcagtgagattgtggtgtgctgtcattctttttatctttttgttatcaaatataaatataacaacattggtgtagagtccactttttgtcctaatatataaacacatctgttttctaaaccagttttatcctttacagggtcacatggctgccagagcttaacctgacaactgatgggcgaagccagggtacaccctggacatgttgccagtctgttggccaaaaacagtctgtgcagggccacaatcacatacaaataggggcaaattagcatgatcaattcaatcaagtctgtattagcatgtggttaacaaatcccaacagtggagcaacatctcacccatgtggtatcaaacagtcttctttttactgcttagattttaattattggttttaaattttgtttcatttattattggtttttcatttttgtgtatgcagttatatctgtttgctcattagttaaataggtgtcttacttaacttttgatttctattccctctcgatcatcttccctagtctggaaaagacagacctgaaagaggtaaagcttatgtaatgtactttgagttgcgaagtagctctgtcttgggctaactagagtgtacatgtaaatgtacctaataaagtgatttgtaaggtacgctcactcagttcgtgaacacactccctcagttatctttgcgcttgttcaattaatggcagtcatgaaacgccatagtcccaactgagatttgaaccggaccttcacgctgtgaggcaatagcgcttttcagtagaaatgtttgctacttcttgtaatgcagaactttgtgattgctagttgtctgccatcaaaaaggctgtatggcggtgtagaggttagctctttcaacttaaagtaagaagaagaacctggttcaaatctctgttggtacctttttgtgtgaagtttggacgtttttcctctgcagatgtggattttctcctgcttactcccacagttcattaataggtgtcatagtataactggttcctgcaaattgctcctgtgtgtttttatggccctttgacaaactggtgacatgattaaggtgtactccacccacttcctgggttggaaccagcaaactggtgactccaaaagagattcagagggcctgaagatgaaaggatggatgcaaaattgcagtggttatttttaaataataatcccttaatggacatccatgtgcaggagacctctatatgcagatatttcttagaaaactattgttagacatctaacagagtatctagacatcctgtggctagcgggatttaccagtcagccagcatgttggtcactgtgaaaatgtctgaacaccatagtgttcggtggagttgaggtcagatctggtgctacgtcattctccttcactgcccaaattaaaaggtagtcctttttatagagtggttacatgtggttacaaatgaaattttgctgggacatataaatctgtgcttatgtgcccattcctagtgctccatgcaattgtggaacaaggggagtatcatcttgatattcttcattgttcaaatatgtgacgtgcaaaacctgaggtggaaaaacaacagtcaacaaggctgcatgcgtgcttgtatttgcagatcgaatcatcttaattctatagagacaaagtgctctggttggctgctttgtgatgattaaaaactgttaaattagcagcaatcagtctgaacaactttgagttgagagcaaagacacgcagtgcaaactggagctcaaaagccaggttatcagtttattgtgaagaagtgaagacaaactttgaaactagatcagacagactttgtgtcagtgtttgcacacgacatcatttgcaagacatttacacaaaacatctattaaaaaggtttgttcatcctctagatgacttgaataacactacaaagtggaaacataaagtgacacttcagtgtcttttcatgatctgttataacttcagaattaatataaaacttaatcatctttcccaaatggatgcgccttaaaaatgtgtcacattttaagagaaataaactggttttccaaccatatgagatctattatcaaaacgtattgtttcaaccatgaaaaaatcctttttttttttggactataagtcgcactttagggagaaatgtaccttacaaaacattgcagaatgaatggggattttatcttgagagacaaataataaaagaataggtaaaaatgacagactggatatgttcctcccagtgttttcccgcactgactgggttgtttgggtggaacccgctgccgcttggacgggttttattttctgttattgggagtgttcaacagggttttcacgggtctctttcatccgcttcactggtccgacggatcagccgctgacagcagcatctcctctcccacctcccttgtggccgaccacgtgtctgcgcaggccctcctgggacttggacgcctgttgggactcgtgactatctacccctccctgcctacctgcagtagtgactgattgtactcaagttcacttgttgtctttttgtttctttaattttgtggtcacactctgcgtgtgagtggggtattggttctgttttgggtttgagtaccagctctgtttaaaaggtgggcttgaagctcctatagggttttattttttttattttaatcctgtttttaaatctatttatgataaatcttttaaatttggaatcacgtctcgacctgattgagtccccatttttttattattgtgcctgaactgctcgtgttgccacatatgctttactactgtagcatattgaggctttttcacgaaacatagaaggaatgtgtcggatctgataaattctaccaaggccggcatgttttcttctgcgttgctgtcagtagcaaatagtgatacaaacacctacagcgccctctattggtttttgctatcacaaaaaaagtcccactggggtataagttgcatctctggccaaactatgcaaaaaactgtgacttatacactgaaaaaactagttcttaacagaatttttttatttgtgctgatttcatgtttacccttctaatgttaatttgtctatggacatgatggatcggtttttccttttatgagtaaaaataatctctgtttcaatgtatttactcttttaaacacagacctagctgcactaaggcaaagtggttagcatttttgcctcaaaacgagaagtccttggttcaaatcccagccaggacctttctatgtgaagtctgtatgttcttcttttgcatgcaggtcactccggctttcttccacagtccaaaaatatgtttaatagattaattgatgtttctaaattgccccaaggtctgcatttgggacaggctgcaaacctgtccaaggtgtaccctgactttgctcaacagtctggtacaggctcagacagccctgtgattccgaaacgggtacagtgggttaaaaaaatgtgtggatggatggaaacccagtcacacagaaatattgtaaaatgagaatagttttcaagctaacagatcttttgttaactatataagtttacatattggtaaattgtagcattttcatccatttctataagtgctacattcaaatccacatgaattagaggaggtaagacatagccatagtatagtattttttgacagctgaaacacctgtggataatcaatgaaaatgaaatttaaaaatggtgtcttcctgactgtatttgttttgttattgtcgatacgactggtgcgatgacaaatcgcttggctctttaaagcaagtgacaagagccgcattcttcctcttaggaaaagttttttttaactgtccttcactcgttattacttctttttctcttcgttattggtctaaatgagataagagccgtttcgttctcgaccgacacatcacagttttgttcagtatgcctttcctgttgctgaagccaaaaataaaaatgactggatttttttctaaaaaaaaaaataaaagctccaaaggcaggtgtctcaaatttgatttattttgcgaacaaaatttcttttagtagttactttcatcagttttattgtatgtggagtcatgggtcatatgttttaacctgaggctagtgggaagggtgaagggtgggttgggtttttattgtaatattgtgtgtgtgtttttttttttttcttttctaaatgttaaagcgctttgagttacgctatgtatgagaagcgcttttctgaaataaagaaataaataaaatcaatctttatttctatagcacttttcatataaaacaaaacaaaacacaaagtgctttacaccagagaaaaaaaaactagccccaacccacacagaacccctaatcccattcaagcctcccaccccttaaatgatggatataaacatttgggaaagaggctgagtatggacaaaaattgtttgtgtaaaagttaatatatggaaccttcctctctgtgaatagctgcatagacagccatgcagcatcacaggtggggacccagcaacaccacagcaaggtggcgatgcaggttcccatctgagccgcagcggcaaaacagcagcggaaccaaagggagaggatccaactgaggaagctctggaatttaaaatgaagataaaaataaaaaataaagaaaacataattagtaaaagaaagaaataattaaccacaaaatgtgaaataaggaatattcaaaagaattgttagtcccgtagaaattcatataatttaatatagtggataaattaataaatacaaaataaataaaaactattaaaatggtaaaattagctaaaagcctgtttaaaaacattagtcttgagcctttccttaaaagcaactattctctctgcagccctcaggtcctctggcagactgttctataaacaacaaccatagtacttaaaagatgcctctatgtaggttttgctttttacagtcggaatgattaattgactagtcagaggatttcgggttcaaacacacgggttcaaacttaactaagagatctgacagataggaaggcacaaaattgttagtatcttaaagcaggagtgtacatatcctaaaatcaaaaaaactaatcttcttctgaatttaaaaaaatgtacgtggttagccttaatttaattatctcaggataccctcccgaatccggtaggtggcaagattgcacgtggaaacttacaaaatgcaaagaaaattagacaattgcggcgaagaagaatcacaaacagtgggcggagcgcaacgcccttaactccgcctacgtgagccacaactcagctcgagccggtggtgtgagcggcctaatcaaacaaacatgacacaggtgtgctcctcgtgccctcagggctctgttactgatgtaggtatttgtaaacggacagctagagctcagctgaaaagaagtgtaattctattcgaacgtagtctttaaaaaaaaatgaaggtgccagaggcggaattaatgagcgacattctgaagaggctgacgggagagtctgctctgccgctgtactgctgcatcgagaagttcaagcgcgagaggaacggcctctactttgtcgccgaggatttcactgaaaccgtcaaaaaaagagaaatggtcaacgccaaggaaagactgagagtgagttcagtttagagaccaaaaatgatccatatttctaatttaaaaacattattaaacaaagcgaatatcagacataatatttgttgtagtatgacagtaattaatgtcattattgctcagtgcagaaagtttgggaacagtagtttcattatcttcagagcccctccgggtccaggagggacttggcctttttctcagtcctcctttctcatcctgcactcgtctttgagaagccgatctaaccatgccgcgcatttggagcttctgtctaccatcaactatgcattgagcaaagacacctggagaggcttcagttcaaacattgtgttcttggtgtcaatttgactggaaagcactacagcttttaattgctattttatgtatttaaaacctacaattttaaactttataattcagcttttttgagtgaatactcttccattaaaaaccagctgtggttcatctttaataattctgaaattctcaaaacttttatcaatttgatctaagttttgtgtcgcacaatatttccaggctctgcagttatcaccgagttactgctggatatgaaatttgggcctactttctctacagataaggaacttgaacacaatgttctcccggctgaagcgcatgctgcctctaatgcaaccagacaaaaagccaagtaaagttgatacactcaaagcagccactgaatacattcgacttcttcttgctgttttgcgggacactgaaaatgtaagactgtcctccggggcccctgtcaaatcacaaaataaatgctgcaacaacaacgctgatatacaatgttgactgctgtcattttacaacttgtagaacaacactgggacggattttctaaagaatgcaatcacttatggtcagcaggatggcttcgccaatgacctctggagaatggacgatgtgagtatttaaatctgtggctgaaatggtagttttaaccaaacatgtaccttagtagcatcacctttacatcaggtttagcctggaaactctgctccatcgtttggaccacatgtgtcaaagtcgaggcccgcaggctacatccggcccgccagatgattttatattattattaatggctcagcaaagtgtagcgctgataacatatttactacagatctcacaatacagcgcttcagctgcccgacgaactataatggtggcatcattttcactacagatcccagaatcagtagcccgcggtttacatgggcagaatatcttgatcggatcaatggtcgggttaaaatttcaccccgtgcgcaagggatcagaaaacctttttcccgattagaacaaacgttcccatggctcacactttcggtcggaatgaatcatttgggcatgcgcagtagtgttaaaaactcccggatgaggaaatgggtcccgttctaaacaaacagctgccacagacatttattttatgctacagctcagtaatatacgagacgatcttccaaatgtgcttttattaatgtcatgaatagtatgaagcgcctgttcgctcatcgttttatttaattcgtgtgatcagtgctggaagagggttaggacaggctataaacacaggctaacaacattagcctgatggacacaaaatccaggaccataaaacgctgcaatctgcattctggctgtatgtaaatgtctgggaataacatcagtctttatttagtcgggacacaactggaaacacaaaaacaagtgattatttaaacagtttctaaggactgagcaacatttatttctgcttcctcaaagccgctgtgtgtgctggcggttcctcctgagtcctggagctgctaacccagagcggcgggacggcttgcagtctgaattctcccacacgtaacaaaacaaaaaaaaacgcacacgtaacaaagcatcctcctcccctcagacacaaagttattaatccagctcctctgctcactcgggtgccagtggaccgagtgagcatcgggggggcacaaagcgctccttccccggatctccctcatggggggtgacagagaggggagagccagcggggcgagagcggagcggcgcttttcacggggcctccgcagagcagctggtcggtctcgtatgcgctccaaagctttctgtcagcgaaacaccatctatgcgtgacgtaaaccagagcagtagtgacacgcgatcggaatgaccatttacatgctccacgatcggataaacgatcaggataacccacttatctcgatcggaaagaaattctgatccgaacgagtctgatcggggcagactattccgaacggcgcgtttacatgacccattttctttccgatcaggcgttctttccccatgtaaacgcagctattgttcagctgccctgctgaacacttttgctagacccacgatgcacaagagaagttaattctgaaaactgagcctttaaaaactctttggaggcagaattaatgtttactgacattcagtaaacctgcatgtcatctgtggagcgaatgtggctgtaatgaaagaatatactctaagatggtgctatgagacagatggcaaatacactttttactgaaaagctgagcacgtggagtttgcacagcgctctagtatcattgaagaacaaaaaatgaattttgttttgtttgtaacctgctttccgtcaatgtggaaacggcacctgtaaggattcagatggcgctgattgagtgttttggtacaccgaaggcaaagtacgaccttcaggttcacacctccgttcctgcagaaatgccccagctccgtctacatgcagcccgatccttgtgcatgtttgggcgcacgtttctgtgtgagaagctcctctcagtgatgaaaactaacaaaacagcagacaggagtcatctccctgatgaacctctacaatacaggacttcacatcaaacacaaaccaacttgacaacaaatgatgccaggcgtccacctgacaaaatgagacaagagcaaagaaatttgactttattttgcagaaaagcacaaattttatttatatatccaggttttatttgttttgttatgcagcaaatacctattttgaatttttgtagttgtgacaggatatatttttatggagaacaaaatatttcggtatatttaagtttttttctatgaaatcagagtaaagtttttttttttatctttagtcgttttactttatttcaaaggtgtatcattttgacagtttatgtttttatggagagaaaatataaagtatttaaggtttaagttagctcaacccatataacccagatacacttcttttcttaaacatagatatgtggactacttggaccacagaacccatttctgatgtttttgttatgctggtgtcaccatatgggtgacatcagcatgggttcttgtgggtaataggatttctgttgtttagttcagttatttttttctgagtttccactgttagaggaattactgaagtcaaattacttgcagtcatttctgtttcctttggttaacaagctgcaagtgtagttgtcactattactaaagcagatctccaaaagcagttggcaaattctaacaagtttaatttttacaactttcttatttggttttcttttagaatttgactcccttaaatctaaaatggatcctcaaattagtattttgggggttttgttgactgaaaacttatttggtttgcttacttaacttgcagttcctgaacatctgagcatttaactgttattttttatctgtctgacagttcttgaacctgtcagatgatcacatttggaggatgggttcaccatgccagcagaacctgcagcagaggatggagacatgactagactggtgttgcagcattgtgtgatgcctgcataccagttcatcatccaagtagcgcctgatcaagcttcggtaagtaaacaattaaaggcgtgggacttgatgttttaaggtgcaatttgtttgcatgaaatgacaacgtatcatttaatccttcaacactggacctttgagctccattgtctacagaattcatatattttggtggattctgaagtggaaaaaaacggctttgtgtcggtgtgcaactttttaccataatagtgtgtagcacaaagtgcatatcggcggtgcacagctaacatacaaagccactttttttctgtctgaatcagctgattcacactaaatggttgaaaggtatggcagatggtgtgttatgtaggtcttgctggcgatatctccagtgttggagggttaaattggcctttgaattcattgaggccaaaataaaatttattaaaattaagtcgggcctgaactggaataactagtgaacaaagtaaaaccataatcttaaaataaaatatgaatttatatcattttaatcaattgtgaatgtgtttaaatcttttcagtggcagccacgaatcaaattctatacagtgatccctcgctataacacagtttacttttcacggtatcgctacttcacggatttgcatcgtgcattgagttctgcattctgattggctaaaaagtcactccccttcttctacctgtgcatcaataacgttgcagtttaatatgtgcacgtacgtaaaacagctcgccaaatttacattatgtacgtgcaaattttctttctggtggcatgtcggtgtataaggatcttttggcaaagaagaaaaaagagcgacatctgcctatcactacgttcttctccctaacaaacacagctgcaccgcgggcttcaaaagaagaaaacactgcagagcggagtcaggatgcagcggctcagtctgaagggcagttgaaatacacctgagtcactatttgtcccgactggaccttggaatttttttcataatcattttaaaattttctccagtttaatcctattactcgggtccgcggg

SEQ ID NO: 23 - DNA, 4067 bp, Oryzias latipes
  misc_feature: Genomic DNA for gene 8
ttgccaattttttttattatgttcaaagttggaaagaaaaacaaaatacattttaatagcactttcatttgtgacatgaaaagctagaaatttacagattgcgagttatttcaagtacaggcatgttttattgccaccttgtggacttttatggtaatgacctgccaggtggacctaaactggttcgattaatatacagtccataaaaaacagtcataatcaacaataagagtgaacagtaaacaatcattggtctcttctattcgcccctgtctcgcggtacatgcgcacttacaggggcggggaggggcccctgattgctgagctcctacaggtgtgcttgagcagcaggtttaatcgcctgcagctgctcggactcttttcagtgcgcagggcctcgtcgctccttccaaactctgaggatggactgcaacctgcggctggccgtttcttgctggatcatggtcttgtcttgggtttcccctctgacagagagtcgccagacgaacagccgaggttctacagaaaggcacttccaacctccagtcgggacgcacggcggtctgcagcctcggctctactcggtgaagcagcagccggccccggaggtccccgaacagcaccgccccgtcacggtcatatgccacccggattccatggaggttgtggtgaaggccgacatgtttgaaacgggcctgaatgtggacggtggacatctgcgactgggttccaacactctgggcgcgggcggtgagtgcggggcggtccagaaaggagaggacgaattcaccatctgggccctgttgtccgactgcggaaccaaactctcagtaagtttcgaacacagcgagcatgcgcctaatgagtccagcatgaaaagactgtcttcttagtcaacagaagagaagatcatttattccaacgttctgatctactcacccgaaccttctgctgatgggttgttaagattggaagctgcaactattccagttgaatgtcattatgacaggtgagtcctgcgtgcatatttatacgcacacctattttgtgcaacagtggccctatggaagagtgtatgccctgagactagaacgtcgtgagcctcaaatccatgagtcatactggtacccaaatcctacctgcttgactctcagaatcaagggattggattgggggtttaaactgccaaatggttcccaataatggctgtctctgcagcttcccatttcccatgcctggctgtattgacgactaatggaacattaacattaaattttattttctctataggagatactctgttgatggcatttcccttaaatcaacttgggttccctctgtctccacaacttctgtgaacgaccagatagatttcaatctgaaactcatgactggtaatcaggggtggctttggaaattatattttgtctgttcaaagccaatatgcggcttcaacaccctgattgtttcaattgcacaggtgactggcagtctgagagggagtcttacacatatttcctggctgatcccattaattttgaagtctctgccatagtggaaaatcacgtccctctgcgggtgtatgtggaccactgtgttgctacggcaactcctgatgcagaggctaatttaagatatgaatttattgaacataaggggtgagcttaaagtcaagcattctgaaagttacttttttttgcctttattactcatctgacatttctgccaaactagctgcctcgttgatgcttaccttacaaactccggagtacgtttcctaccaagaaccgaggaacataaactgaggtttcagctggaagccttcaggttctatcaagaacccagcaaccaggtgtggctccaaatgaaacatttgtacgcttatgaaagttttcatactgcttaaccatttttgtcgttgcacaacacaaggtaactcggacactttgtgaactcaaatcttggcgttctgcgtagttccccctttggcatatcccttcagggggcgccacagcaaatcagccttcactgagtcacacaggtggtttggcagagttttatgccagatgcccttcagacaaccatgcatttttacctggcccatttttatttgctggttatagttcagcagtagcgcaaagggtcttgcccacggacccatgctggatgaagcttattgtgttccatgggaattgaaccctgattccccgtgtgccttaagcaattggacgatcagtcactatagtcggtttaattattgtgcttgatccaaaattattgctgactggtttgggaaaactgcttgtgagtcaccacttttccctattgtttccagatttacattacttgtgctgtgaaggctgttcctgctgtacaggcggtcagttctcagaaccgagcctgctcctttattgagaacaggtgatctattgaaatagaaatgcaaaaatgttctagtgcttgttgcacaaatcttaactgctaccatctcgctccagatggcaatccatagacggtggtgatcaggtgtgcagaagctgtgacgtgtccaggcggggtcaggaaccgcaagctgtgccatctcctaaaatggcagtgaacgccaaagaccaaatcggtctttcacagaaaaatatagtccacaataaagccgagcatcaaccggcttcttacgtccatttttggccgggagcatatcagagccatcactccaaacctcagcagtccaccaacagatttatgaagagggatgctgacaacaaatttcgtgagtagaacttaaagggcctatttcatgcaaataaactttttgagcttttaaattgttagttcctcacaaaaaacaactccaaagcagtattttgctacagtcacgcatttctgagcattcctttaaaaacctgctctgagcaccagcccctcccaatccacaaaaacacattgtgagcgaggaacagccccttccatgaagagtctgcgctgccagcaccgcccccaggctaacccacacctactttatacatggagctagcgttggttgatcggcaaaaacgtttttattgtcatatgctcagttgtctctgcaaaagttcagacgttttcgtgggcgaaacagacatgctgcggctctaggttgattgtgaaaagggcggcacccacacggagtgaaagtcgttgcatcagatcgagtattacttctgggtaggaaaatgatctgagaaaatgactaatttcataaatttgtttttttttgagtctgctaaaggttatacatatggatacacttgactctttaaaggcttgataaaagcatgaaatggccccttcaattaaaataaatctgaagagtttgtgttaatttaatgtttgtctattgtaaaagctggaaagtatctaaaattgactcttggttttgaaaatgtcttactttgcagatcaagctgtccaactggggcccctcgttgtgctaccgtcaagaaaagtagtttcagtggcaacaaatttttcaacatggtcagaaaagaacacctcctgagacctggaacacctgatcgaggagtctgagttcatttgagaaatttggtcttgatgggctaaagaattttgctgcaatccaataaattaataaatttctaatgtaaactttatttgtaaaaatgatgctgcttattctgtggaccactctttctccagtaaatattactggtttgtgtcccctttaaatgtatataatatcgactaaagcaatttgtgtggagaagctgttggtccatggacttcataaaactaaagcgaacaacttcattgttattgtatatcaggcacttggcctcattaaacttttggaacataactagatattggattatgacaaattgaagacgactggagaaacgtgatcctagctcatgtctcttgcagggacatgctattagggagcatttccttccattactgccacacctaatcatgtagtcacagtattgattatctgaaggctatgatgtaacgtgtttgcttagcttgagtgatcaattacagctttgccttaattattctggaacctt

SEQ ID NO: 24 - DNA, 23 bp, Artificial
  PCR primer 863.3
gtacaagcgcgagaggaacggcc

SEQ ID NO: 25 - DNA, 24 bp, Artificial
  PCR primer 863.1
ttctccagaggtcattggcgaagc

SEQ ID NO: 26 - DNA, 20 bp, Artificial
  PCR primer 1/15
gatcaggcgctacttggatg

SEQ ID NO: 27 - DNA, 20 bp, Artificial
  PCR primer 6a
ggagatactctgttgatggc

SEQ ID NO: 28 - DNA, 20 bp, Artificial
  PCR primer 6b
cgtcacagcttctgcacacc

SEQ ID NO: 29 - DNA, 18 bp, Artificial
  PCR primer 8.3
agactcctccatcaggtg

SEQ ID NO: 30 - DNA, 21 bp, Artificial
  PCR primer F1
tccttccctgtttcgtcttgg

SEQ ID NO: 31 - DNA, 21 bp, Artificial
  PCR primer R1
ttgcaggtggattcacagcag

SEQ ID NO: 32 - DNA, 21 bp, Artificial
  PCR primer F2
gctttcctgcgactatcagcc

SEQ ID NO: 33 - DNA, 21 bp, Artificial
  PCR primer R2
atttggatcccatgcaaccag

(19) United States
(12) Patent Application Publication
(10) Pub. No.: US 2003/0162187 A1
(43) Pub. Date: Aug. 28, 2003

(54) Method of detecting sexual differentiation disruptor

(76) Inventors: Akira Kanamori, Nagoya-shi, Aichi (JP); Masato Kinoshita, Kyoto-shi, Kyoto (JP); Ryokichi Takashima, Otsu-shi, Shiga (JP); Hideto Chono, Moriyama-shi, Shiga (JP); Shigetoshi Mizutani, Gamo-gun, Shiga (JP); Akiro Kondo, Kusatsu-shi, Shiga (JP); Ikunoshin Kato, Uji-shi, Kyoto (JP)

Correspondence Address: BROWDY AND NEIMARK, P.L.L.C., 624 NINTH STREET, NW, SUITE 300, WASHINGTON, DC, 20001-5303, US

(21) Appl. No.: 10/169,157

(22) PCT Filed: Dec. 25, 2000
(86) PCT No.: PCT/JP00/09170

(30) Foreign Application Priority Data
Dec. 28, 1999 (JP) 11/372602

(51) Int. Cl. C12Q 1/68; A01K 67/00; C12N 15/85

(52) U.S. Cl. 435/6; 435/455; 800/8

(57) ABSTRACT

Killifish genes which are expressed specifically to females in accordance with the phenotype sex, characterized by having a base sequence selected from among the base sequences represented by SEQ ID NOS: 1 to 21 in Sequence Listing.

### TECHNICAL FIELD

The present invention relates to a method for rapidly assessing an endocrine-disrupting activity of a chemical substance as well as a series of techniques connected therewith in fields of medicine, pharmacology, environmental study and sitology.

### BACKGROUND ART

Endocrine disruptors (often called environmental hormones) collectively refer to chemical substances released into the environment for which hormone-like activities or anti-hormonal activities have been found. Altered reproductive potential (in particular, conversion of male into female), decreased reproductive potential, decreased hatchability, decreased survival rate of offspring, abnormal reproductive behavior and the like have been reported to be resulted from the influences of endocrine disruptors on the ecosystem of wild animals. Decreased number of sperms, endometriosis, infertility, ovarian cancer, uterine cancer, prostatic cancer and the like have been suspected to be resulted from the influences of endocrine disruptors on human health, although they have not been demonstrated.

Substances (or groups of substances) that are considered to cause endocrine disruption are reported in the interim report (July 1997) by “Exogenous Endocrine Disrupting Chemical Task Force” of Environment Agency. However, it is considered that the types of such substances would be further increased in the course of research and study in the future.

Known methods for determining endocrine disrupting activities are classified into two groups, i.e., in vitro methods and in vivo methods. Examples of the methods in the former group include a method in which an activity of binding to estrogen receptor, androgen receptor or thyroid hormone receptor is measured, and a method in which an activity of inhibiting a hormone synthesis enzyme system is measured. Examples of the methods in the latter group include a method in which production of various hormones and abnormal tissue formation in individuals at different postnatal days are determined, a method in which abnormal metamorphosis in a frog is determined, and a method in which abnormal maturation in a fish is determined (Analytical Chemistry, 70(15):528A-532A (1998)).

The in vitro method is advantageous because it is sensitive, and it can be used to assay a number of test samples in a short time. However, it cannot be determined whether or not endocrine is actually disrupted using the in vitro method because it only determines an activity of binding to a receptor or the like. On the other hand, the actual influence on endocrine is directly examined using the in vivo method in which animals such as rats, frogs, fishes or the like are used. Thus, such a method is necessary in order to determine the influence on a living body or the environment. However, the in vivo method has drawbacks because, for example, its sensitivity is low, it requires complicated operation and, if a number of samples are to be examined, it requires a long time.

For example, a system in which conversion of male into female is monitored to assess an endocrine-disrupting activity has been constructed. The monitoring is carried out by determining the expression of a fish female-specific yolk precursor protein vitellogenin in males using an anti-vitellogenin antibody. However, it is difficult to deal with a number of test samples at a time using the system. The sensitivity of the system in which adult fishes are used for the assessment is supposed to be lower than that of an assessment system in which fries are used because fries are relatively subject to disrupting activities. The system has further problems because it requires a wide space for breeding and a long breeding period. In addition, anti-vitellogenin antibodies specific for the respective fishes to be assessed are required for the system.

An assessment method in which the hatchability of eggs or the number of eggs spawned from an adult fish are used as indexes, and an assessment method in which courtship behavior or the like is monitored have been proposed (Lisa, D. et al., Environmental Toxicology and Chemistry, 17:49-57, 1998). However, it cannot be determined whether or not sexual differentiation is actually disrupted using such methods.

Disruption of sexual differentiation can be examined by determining both the genotypic sex and the phenotypic sex of an individual and comparing them each other. For example, a system for assaying a disrupting activity in which sex reversal is used as an index has been proposed. In the system, a fry or an egg is bred while administering an environmental hormone thereto, and the phenotypic sex associated with secondary sex characteristics is then determined. For example, in case of medaka, the genotypic sex can be determined by using a PCR (Shinoyama, A. et al., The Fish Biology Journal MEDAKA, 10:31-31, 1999), or a medaka strain of which the genotypic sex is linked to pigment expression, d-rR (Yamamoto, T., J. Exp. Zool., 123:571-594, 1958) or Qurt (Wada, H. et al., Zoological Science, 15:123-126, 1998). It is required to prepare a tissue section to microscopically examine a gonad in order to determine the phenotypic sex of a fry. Thus, it is difficult to deal with a number of test samples. Breeding for at least one month is required in order to determine the phenotypic sex based on the shape of a fin associated with secondary sex characteristics. Furthermore, skill is required for the determination. It is also difficult to deal with a number of test samples using this method. As described above, in fact, the in vivo assay requires a long time from the start of assessment, and it is difficult to assay a number of test samples at a time. However, such an in vivo assay is necessary for assessing chemical substances or water environment, or monitoring water pollution. In addition, construction of a rapid and accurate assay system has been desired.

Examination of an endocrine-disrupting activity may provide an index for assessing the influence of a chemical substance on humans, for determining the influence on living bodies upon its release into the environment or on the ecosystem, or for monitoring water pollution. However, the prior art has drawbacks as described above. Thus, a sensitive and rapid method for determining an endocrine-disrupting activity has been desired.

### SUMMARY OF THE INVENTION

As a result of intensive studies, the present inventors have successfully isolated and identified genes expressed specifically in phenotypic females of medaka during early development for the first time. The present inventors have found that disruption of sexual differentiation can be examined by rapidly determining the phenotypic sex for a fry of medaka using the gene, and comparing the phenotypic sex with the genotypic sex. The present inventors have also found that an endocrine-disrupting activity of a sample can be rapidly determined using the method. The present inventors have successfully constructed a method for rapidly assessing endocrine-disrupting activities of chemical substances, samples of water environment (lakes, marshes, rivers, seas, etc.) or the like in vivo for a number of samples at a time. Thus, the present invention has been completed.

The present invention is outlined as follows. The first aspect of the present invention relates to a gene expressed in a female-specific manner depending on its phenotypic sex, which has a nucleotide sequence selected from the group consisting of the nucleotide sequences of SEQ ID NOS: 1 to 21. It also relates to a gene which hybridizes with said sequence under stringent conditions as well as a gene which has said sequence as well as an intron or introns being inserted.

The second aspect of the present invention relates to a method for assessing a sexual differentiation-disrupting activity of a sample, the method comprising:

(1) administering a sample to be assessed for its sexual differentiation-disrupting activity to a medaka;

(2) determining the genotypic sex of the medaka;

(3) determining the phenotypic sex of the medaka based on the expression of a female-specific gene; and

(4) determining if the sexual differentiation is disrupted based on the results of steps (2) and (3).

The third aspect of the present invention relates to a method for detecting an endocrine disrupter, comprising assessing a sexual differentiation-disrupting activity by the method of the second aspect.

The fourth aspect of the present invention relates to an oligonucleotide for detecting the gene of the first aspect.

The fifth aspect of the present invention relates to a kit for assessing a sexual differentiation-disrupting activity by the method of the second aspect.

The sixth aspect of the present invention relates to a kit for detecting an endocrine disrupter by the method of the third aspect.

### DETAILED DESCRIPTION OF THE INVENTION

The present invention is described in detail below.

The present invention may be applied to any samples without limitation, including naturally occurring or artificially synthesized chemical compounds. Such a substance may be subjected to the method of the present invention in an isolated form or in a mixture. The method of the present invention is applicable to a sample from the environment such as river water, soil or the like.

A medaka (Oryzias latipes) is used as an organism according to the method of the present invention. It is widely used as a material for studying genes because it is small and easy to handle, it releases a lot of eggs upon spawning, its generation time is short (about three months), and a genetically homogeneous strain can be established by inbreeding.

A medaka can be bred in distilled water in a 96-well microplate for about one week after hatching. Since feeding is unnecessary, secondary factors such as a disrupting activity due to a bait can be excluded upon assessment.

One can breed a medaka in water containing salt at various concentrations, including fresh water and seawater, at a wide range of temperatures from about 0 to about 30° C. Thus, it can be used for assessment assuming various environments.

There is no specific limitation concerning the medaka to be used according to the present invention. A wild type medaka or a medaka strain of which the genotypic sex is linked to pigment expression such as d-rR or Qurt may be used.

Qurt is a medaka strain heterozygous for the leucophore free (if) locus, which is closely linked to sex. A female (X^{lf}/X^{lf}) individual of Qurt is colorless, whereas a male (X^{lf}/Y⁺) individual is yellow as a result of pigment expression. The yellow color can be observed for an egg. Therefore, Qurt can be preferably used according to the present invention because its genotypic sex can be readily determined by microscopically examining the egg without extracting the DNA (Zoological Science, 15:123-126, 1998).

There is no specific limitation concerning the method for determining the genotypic sex according to the present invention. For example, the genotypic sex can be determined by genetic analysis of sex chromosomes.

The genotypic sex of medaka is fixed upon fertilization depending on the combination of sex chromosomes as follows: female in case of X/X; and male in case of X/Y. Thus, the genotypic sex can be determined by genetic analysis of sex chromosomes. There is no specific limitation concerning the method for the genetic analysis. For example, the analysis can be carried out by hybridization using a probe that hybridizes with a gene on the sex chromosomes or a PCR using primers that can be used to amplify a gene on the sex chromosomes. If such a method is used to determine the genotypic sex, it is also necessary to prepare both DNA and RNA from an individual. This is because it is necessary to analyze the expression of a female-specific gene using RNA for determining the phenotypic sex as described below. In this case, the head portion of a fry is cut off. The remaining body portion which contains a gonad and the like is used to determine the expression of a gene that is specifically expressed depending on the phenotypic sex as described below. DNA extracted from the head portion is used to determine the genotypic sex. Alternatively, DNA and RNA may be prepared simultaneously using QIAGEN RNA/DNA System (QIAGEN). Furthermore, DNAs can be prepared simultaneously from a number of test samples in a 96-well microplate using DNeasy 96 Tissue Kit (QIAGEN).

If the medaka strain Qurt is used, the difference in pigment expression specific for the genotypic sex can be recognized on the second day after fertilization. Thus, the genotypic sex can be determined for an egg by detecting the pigment without preparing DNA.

A gene that is expressed in a medaka in a phenotypic sex-specific manner is used for determining the phenotypic sex according to the method of the present invention. For example, a medaka gene expressed in a phenotypic female-specific manner is used. A gene that is specifically expressed within five days after hatching is preferable for assessment at an early stage.

Examples of such genes include the following:

(1) FIGα, a transcription factor containing a basic helix-loop-helix motif;

(2) eIF-4, a cap-binding subunit of an elongation initiation factor;

(3) genes encoding ZP domain-containing proteins;

(4) 42Sp50 and 42Sp43, genes encoding oocyte-specific RNA storage proteins;

(5) quinone reductase gene; and

(6) unknown genes encoding secretory proteins.

Such genes are exemplified by ones having the sequences of SEQ ID NOS: l to 21 or sequences that hybridize with said sequences under stringent conditions. Stringent hybridization conditions include, for example, those as described in T. Maniatis et al. (eds.), Molecular Cloning: A Laboratory Manual 2nd ed., Cold Spring Harbor Laboratory, 1989. Incubation with a probe at 65° C. overnight in a solution containing 6×SSC (1×SSC: 0.15 M NaCl, 0.015 M sodium citrate, pH 7.0), 0.5% SDS, 5× Denhardt's and 100 mg/ml herring sperm DNA exemplifies the conditions.

The gene may exist on a chromosome with an intron or introns being inserted. The present invention also encompasses such a gene having an intron or introns being inserted. Examples of such genes include a gene having the nucleotide sequence of SEQ ID NO: 22 (the sequence of SEQ ID NO: 1 with introns being inserted) or a gene having the nucleotide sequence of SEQ ID NO: 23 (the sequence of SEQ ID NO: 8 with introns being inserted).

For example, the above-mentioned genes can be isolated as follows.

The sexual differentiation of a medaka is first manifested as a phenomenon that the number of germ cells in a female is about twice as many as that in a male upon hatching, i.e., on about tenth day after fertilization (Satoh, N., Egami, N., J. Embryol. Exp. Morph., 28:385-395, 1972). This is because mitosis is initiated immediately after hatching in a portion of germ cells in a female whereas germ cells in a male do not divide until two months after hatching. It is possible to identify genes expressed in a female-specific manner at an early development stage using the difference in sexual differentiation in germ cell line as an index. Difference in gene expression between a male and a female of medaka can be examined using subtractive hybridization. Genotypic male and female are separated each other before hatching. RNAs are extracted from the both after hatching. Then, a gene expressed in a female-specific manner can be isolated using subtractive hybridization. If the medaka strain Qurt is used, genotypic male and female can be readily distinguished before hatching on the second day after fertilization using the expression of the pigment gene as an index.

A genomic gene corresponding to each gene can be isolated by screening a genomic library according to a known method using the thus obtained gene as a probe.

The gene can be detected using an oligonucleotide designed based on the nucleotide sequence of the gene. The oligonucleotides for detecting the gene according to the present invention include, but are not limited to, primers that can be used to amplify the gene or a portion thereof according a gene amplification method, and a probe that is hybridizable with the gene under stringent conditions.

Examples of gene amplification methods that can be used include, but are not limited to, PCR, SDA, NASBA and ICAN (WO 00/56877).

A primer or a probe can be designed at will based on the nucleotide sequence of the gene. Of course, a sequence is selected upon designing such that the primer or the probe does not form a secondary structure within the molecule, and attention is paid such that the melting temperature (Tm value) for the primer or the probe and the corresponding template is set at an appropriate temperature.

The Tm value of a primer or a probe can be determined, for example, according to the following equation:

Tm=81.5−16.6(log₁₀[Na⁺])+0.41(%G+C)−(600/N)

wherein N is the chain length of the primer or the probe; % G+C is the content of guanine and cytosine residues in the primer or the probe.

If the chain length of the primer or the probe is shorter than 18 bases, the Tm value can be estimated, for example, as the sum of the product of the number of adenine and thymine (A+T) residues multiplied by 2(° C.) and the product of the number of G+C residues multiplied by 4(° C.), i.e., [(A+T)×2+(G+C)×4].

Although is not intended to limit the present invention, the chain length of the probe is preferably 15 bases or more, more preferably 18 bases or more in order to avoid nonspecific hybridization.

Although it is not intended to limit the present invention, for example, a primer of 15 to 40 bases in length can be used. In particular, a primer of 17 to 30 bases in length can be preferably used.

Furthermore, it is desirable to design a primer such that the ratio of cytosine (C) and guanine (G) around the 3′-terminus becomes high. A commercially available software for primer designing such as OLIGO™ Primer Analysis software (Takara Shuzo) may be used for designing a primer.

The primer or the probe may have a mutation such as deletion, substitution, insertion or addition of a nucleotide (or nucleotides) in a portion of the sequence as long as it can be used to detect the gene. In case of a primer, it is preferable not to include a mutation or, if any, to minimize mutations around the 3′-terminus of the primer because such a mutation greatly influences the efficiency of primer extension reaction. An appropriate sequence unrelated to the nucleotide sequence of the gene (e.g., a promoter sequence recognized by an RNA polymerase) may be added on the 5′ side of the primer. optionally, the primer or the probe may be appropriately modified. Addition of a ligand such as biotin or digoxigenin, or a fluorescent substance to a primer or a probe facilitates the detection of the amplification reaction product.

A product of a gene amplification reaction using the primers can be detected by subjecting a portion of the reaction mixture after the amplification reaction to electrophoresis, and then staining the DNAs with ethidium bromide. An amplification product can be detected without electrophoresis by utilizing hybridization. If a modified primer is used, a detection method suitable for the modification can be used.

A kit containing the primer or the probe can be constructed and used for detecting the gene according to the present invention. Such a kit may contain a buffer or an enzyme to be used for an amplification reaction or hybridization. It may further contain a reagent to be used for preparation of a nucleic acid sample from cells or detection of an amplification product in order to make the detection more convenient.

There is no specific limitation concerning the method for detecting the expression of the gene. For example, the expression can be detected by detecting the mRNA transcribed from the gene in RNA prepared from a medaka on the 1st to 5th day after hatching by Northern hybridization, RT-PCR or the like.

RNA can be prepared from a medaka, for example, by directly treating the medaka individual using TRIzol reagent (Gibco-BRL) or the like. Alternatively, RNA and DNA can be simultaneously prepared using QIAGEN RNA/DNA System (QIAGEN). In this case, the DNA can be used for the determination of the genotypic sex. Furthermore, DNAs can be prepared simultaneously from a large number of test samples in a 96-well microplate using RNeasy 96 Kit (QIAGEN).

In order to exclude false positive results due to products amplified from a genomic DNA, it is desirable to use a pair of primers for RT-PCR that can be used to amplify a region of mRNA transcribed from the gene of interest and that is designed such that the corresponding region in the gene contains an intron or introns being inserted. For example, a combination of a primer F1 (SEQ ID NO: 30) and a primer R1 (SEQ ID NO: 31), a combination of a primer F2 (SEQ ID NO: 32) and a primer R2 (SEQ ID NO: 33) or the like can be used to detect Gene 5. The primers F1, R1, F2 and R2 have sequences in the exons 2, 8, 2 and 7, respectively.

If the primers F1 and R1 are used, the size of the product amplified from the mRNA is about 730 bp, whereas the size of the product amplified from the genomic DNA is about 1.1 kbp. If the primers F2 and R2 are used, the size of the product amplified from the mRNA is about 590 bp, whereas the size of the product amplified from the genomic DNA is about 0.9 kbp. Thus, the product amplified from the mRNA can be clearly distinguished from the product amplified from the genomic DNA as a background.

More sensitive detection can be accomplished using these primer pairs by carrying out a 1st PCR using the pair of primers F1 and R1 followed by a nested PCR using the pair of primers F2 and R2.

A combination of a primer 863.3 (SEQ ID NO: 24) and a primer 863.1 (SEQ ID NO: 25), a combination of a primer 863.3 (SEQ ID NO: 24) and a primer 1/15 (SEQ ID NO: 26) or the like can be used to detect Gene 1. The primers 863.3, 863.1 and 1/15 have sequences in the exons 1, 3 and 4, respectively. If the primers 863.1 and 863.3 are used, the size of the product amplified from the mRNA is about 300 bp, whereas the size of the product amplified from the genomic DNA is about 1 kbp. If the primers 863.3 and 1/15 are used, the size of the product amplified from the mRNA is about 400 bp, whereas the size of the product amplified from the genomic DNA is about 4 kbp. Thus, the product amplified from the mRNA can be clearly distinguished from the product amplified from the genomic DNA as a background.

A combination of a primer 6a (SEQ ID NO: 27) and a primer 6b (SEQ ID NO: 28), a combination of a primer 6a (SEQ ID NO: 27) and a primer 8.3 (SEQ ID NO: 29) or the like can be used to detect Gene 8. The primers 6a, 6b and 8.3 have sequences in the exons 3, 7 and 8, respectively. If the primers 6a and 6b are used, the size of the product amplified from the mRNA is about 530 bp, whereas the size of the product amplified from the genomic DNA is about 1.3 kbp. If the primers 6a and 8.3 are used, the size of the product amplified from the mRNA is about 880 bp, whereas the size of the product amplified from the genomic DNA is about 2.2 kbp. Thus, the product amplified from the mRNA can be clearly distinguished from the product amplified from the genomic DNA as a background.

In order to exclude false positive results due to a genomic DNA, it is desirable to use a probe for Northern hybridization that hybridizes with a region of mRNA transcribed from the gene of interest and that is designed such that the corresponding region in the gene contains an intron or introns being inserted.

The expression of the gene can be detected using a DNA microarray. A DNA microarray is a material having nucleic acids being immobilized in which a number of different genes or DNA fragments are arrayed and immobilized on a solid phase substrate such as a slide glass. The DNA microarray is used to examine the existence of a nucleic acid in a nucleic acid sample that has a sequence complementary to the DNA immobilized on the microarray by contacting it with a nucleic acid sample (preferably a labeled nucleic acid sample) prepared from a sample for hybridization. Expression of plural genes specifically expressed depending on the phenotypic sex can be monitored at the same time using the microarray.

Also, the expression of the gene can be detected using an antibody to a protein translated from the gene. There is no specific limitation concerning the antibody used as long as it can recognize the protein expressed from the gene. A polyclonal antibody, a monoclonal antibody or the like prepared according to a known method may be used.

A sexual differentiation-disrupting activity of a sample can be assessed by comparing the phenotypic sex with the genotypic sex both determined as described above. An endocrine-disrupting activity of a sample can be assessed according to this method. Although it is not intended to limit the present invention, disruption of sexual differentiation may be represented by expression of a phenotypic female-specific gene in a genotypic male individual, or loss of expression of a phenotypic female-specific gene in a genotypic female individual.

For example, a sexual differentiation-disrupting activity of a sample can be assessed as follows. A medaka egg immediately after fertilization is bred in a test water sample. The genotypic sex and the phenotypic sex are determined for each individual as described above. An individual is determined to be influenced by an sexual differentiation-disrupting activity if the genotypic sex of the individual is different from the phenotypic sex of the same individual. The sexual differentiation-disrupting activity of the sample can be assessed by counting the number of such individuals. If a medaka strain Qurt of which the genotypic sex is linked to pigment expression is used, the relationship between the genotypic sex and the pigment expression may be reversed due to translocation of chromosome with the probability usually at several percentage or less. This problems can be solved by increasing the number of test samples. Translocation of chromosome is rarely observed for a medaka strain d-rR of which the genotypic sex is also linked to pigment expression. Thus, almost no reversion is observed for the relationship between the genotypic sex and the pigment expression if this medaka strain is used.

A kit for assessing a sexual differentiation-disrupting activity according to the present invention is one for assessing the activity using the method of the present invention. Although it is not intended to limit the present invention, a kit containing an oligonucleotide that can be used to detect the gene of the present invention as described above is exemplified. Such a kit may contain a buffer or an enzyme to be used for an amplification reaction. It may further contain a reagent to be used for preparation of a nucleic acid sample from cells or detection of an amplification product in order to make the detection convenient.

A kit for detecting an endocrine disruptor according to the present invention is one for detecting an endocrine disrupter using the method of the present invention. Although it is not intended to limit the present invention, a kit containing an oligonucleotide that can be used to detect the gene of the present invention as described above is exemplified. Such a kit may contain a buffer or an enzyme to be used for an amplification reaction. It may further contain a reagent to be used for preparation of a nucleic acid sample from cells or detection of an amplification product in order to make the detection convenient.

The phenotypic sex can be determined at an early stage by the fifth day after hatching according to the method of the present invention. Conventionally, the phenotypic sex could be determined only based on the shape change of a fin associated with secondary sex characteristics one month or more after hatching or the like. Furthermore, no special skill is required for the present invention. Thus, it is also possible to deal with a number of test samples according to the method of the present invention. The method is effective in rapidly and conveniently assessing endocrine-disrupting activities of naturally occurring or artificially synthesized chemical substances as well as samples from the environment such as river water or soil.

### EXAMPLES

The following examples further illustrate the present invention in detail but are not to be construed to limit the scope thereof.

### Example 1

Identification of Female-Specific Genes by Subtractive Hybridization

A pair of mature Qurt medakas consisting of one male and one female (Zoological Science, 15:123-126, 1998) was bred in 3 L of green water (purified water containing chlorella from green algae) at 25° C. under conditions of a light period for 14 hours and a dark period for 10 hours. They were fed with TetraMin three to five times a day setting the amount of TetraMin such that it was consumed within three to five minutes, and then spawned. Embryos were genotypically classified into males and females by examining the expression of the yellow pigment gene as autofluorescence under a fluorescence microscope on the fourth day after spawning. mRNAs were prepared from samples at four stages (stage 37/39 (2-3 days before hatching), or 1, 5 or 30 day(s) after hatching) according the classification table of Iwamatsu (Iwamatsu, T., Zool. Sci., 11:825-839, 1994). cDNAs were prepared using an oligo(dT) primer and Copy Kit (Invitrogen). The cDNAs were cleaved with a restriction enzyme AluI, ligated to linkers (Wang, Z. and Brown, D., Proc. Natl. Acad. Sci. USA, 88:11505-11509, 1991), and amplified using PCRs. Subtractive hybridization was carried out using cDNAs from a male and a female at each stage. The remaining cDNAs were amplified using PCRS. This process of subtraction/PCR amplification was repeated three times. The amplified cDNAs for the males and the females at the respective stages were cloned into a plasmid to obtain eight cDNA libraries. Fragments inserted in clones selected at random from the respective libraries were isolated and subjected to Southern hybridization to screen for genes expressed in a sex-specific manner at each stage. The starting cDNAs and the cDNAs obtained after three rounds of subtraction/PCR amplification were used as probes for the Southern hybridization. No male-specific positive reaction was observed. Thus, no gene expressed in a male-specific manner could be isolated. Three clones and forty-seven clones were obtained from the 5th and 30th day female libraries, respectively, as clones that exhibit positive reactions in a female-specific manner. The fragments inserted in these clones were used as probes for hybridization with the cDNAs from the 1st, -5th or 30th day male or female (both the starting cDNAs and the cDNAs obtained after three rounds of subtraction/PCR amplification). Based on the results of hybridization, the genes were classified into three groups, i.e., groups of genes expressed in females on the 1st, 5th or 30th day. Nucleotide sequences of two genes classified as those expressed in females on the 1st day (Genes 1 and 2) and nineteen genes classified as those expressed in females on the 5th day (Genes 3-21) were determined. The determined nucleotide sequences of Genes 1-21 are shown as SEQ ID NOS: 1-21.

Homology searches of database for the determined gene sequences revealed that the Genes shared homologies with known genes as follows: Gene 1 (SEQ ID NO: 1)—gene for FIGα, mouse transcription factor having a basic helix-loop-helix motif; Gene 2 (SEQ ID NO: 2)—gene for eIF-4, human cap-binding subunit of elongation initiation factor; Gene 3 (SEQ ID NO: 3)—gene encoding rabbit ZPA domain; Gene 4 (SEQ ID NO: 4)—gene encoding goldfish ZPB domain; Gene 5 (SEQ ID NO: 5)—gene encoding carp ZPC domain; Gene 6 (SEQ ID NO: 6)—gene encoding zebra fish ZPC domain; Gene 7 (SEQ ID NO: 7)—gene encoding carp ZPC domain; Gene 8 (SEQ ID NO: 8)—gene encoding zebra fish ZPC domain; Gene 9 (SEQ ID NO: 9)—gene encoding zebra fish ZPC domain; Gene 10 (SEQ ID NO: 10)—Xenopus 42Sp42 gene which encodes oocyte-specific RNA storage proteins; Gene 11 (SEQ ID NO: 11)—Xenopus 42Sp50 gene; and Gene 12 (SEQ ID NO: 12)—rat quinone reductase gene. Genes 13-21 (SEQ ID NOS: 13-21) did not share homology with a known gene. It is supposed that they unknown genes that encode secretory proteins based on the sequence characteristics.

Next, a genomic library was constructed using chromosomal DNA prepared from a medaka according to a known method. Screening was carried out using Gene 1 or 8 as a probe. Corresponding genomic genes were isolated and the nucleotide sequences were determined. Nucleotide sequences of Genomic Gene 22 (corresponding to Gene 1) and Genomic Gene 23 (corresponding to Gene 8) are shown as SEQ ID NOS: 22 and 23.

### Example 2

Detection of Sexual Differentiation-Disrupting Activity of 17 β-estradiol

Pairs of mature Qurt medakas each consisting of one male and one female were bred in 3 L of green water (purified water containing chlorella from green algae) at 25° C. under conditions of a light period for 14 hours and a dark period for 10 hours. They were fed with TetraMin three to five times a day setting the amount of TetraMin such that it was consumed within three to five minutes,;and then spawned. Eggs resulted from five pairs were placed in a Petri dish immediately after spawning, separated each other using tweezers in tap water from which chlorine had been removed and washed. The water was replaced by a 1 -ppb 17 β-estradiol (E2) aqueous solution containing 0.1% dimethyl sulfoxide. The mixture was dispensed into wells of 96-well round bottom microplate (#3797, Corning) which had been extensively washed with ultrapure water such that each well contained one egg. After covering with a lid, the microplate was incubated in an incubator at 25° C. On the third day from the start of incubation, males and females were distinguished by examining the yellow pigment which is expressed in a male-specific manner as autofluorescence using a fluorescence microscope (Nikon) The incubation was further continued, and fries then hatched on the 7th to 9th day after spawning. Fries were collected on the 12th day after spawning (the 3rd to 5th day after hatching), then soaked in RNAlater (#7021, Ambion) and stored at −80° C. RNAs were extracted from the fries using StrataPrep Total RNA Miniprep Kit (#400711, Stratagene). The test samples were soaked in a lysis solution attached to the kit, and homogenized in 1.5 -mL tubes using a pellet mixer (Urin Seisakusho). Then, extraction was completed according to the manual attached to the kit. The extracted RNAs were subjected to TaKaRa One Step RNA PCR Kit (AMV) (Takara Shuzo). 1st PCRs were carried out using a pair of primers F1 (SEQ ID NO: 30) and R1 (SEQ ID NO: 31) which is used to amplify a region of 729 bp in Gene 5 (SEQ ID NO: 5), a gene expressed in a female-specific manner. Next, nested PCRs were carried out using a pair of primers F2 (SEQ ID NO: 32) and R2 (SEQ ID NO: 33) which is used to amplify a region of 593 bp within the 729 -bp region using 0.5 μL each of the products of the 1st PCRs as templates. The resulting amplification products were subjected to electrophoresis on 2% agarose gel. The results are shown in Table 1. In the table, the genotypic sex represents the result of microscopic examination, and the phenotypic sex is represented as the result of distinction between a female and a male using the RT-PCR.

TABLE 1
Control group
(treatment with solvent (water containing 0.1% DMSO))

| | | | | | | | | |
|---|---|---|---|---|---|---|---|---|
| Sample No. | 1 | 2 | 3 | 4 | 5 | 6 | 7 | 8 |
| Genotypic sex | ♂ | ♂ | ♂ | ♂ | ♀ | ♀ | ♀ | ♀ |
| Expression of Gene 5 | − | − | − | − | + | + | + | + |

Treatment with 1 ppb 17 β-estradiol

| | | | | | | | | | | |
|---|---|---|---|---|---|---|---|---|---|---|
| Sample No. | 1 | 2 | 3 | 4 | 5 | 6 | 7 | 8 | 9 | 10 |
| Genotypic sex | ♂ | ♂ | ♂ | ♂ | ♂ | ♀ | ♀ | ♀ | ♀ | ♀ |
| Expression of Gene 5 | − | − | − | + | + | − | − | + | + | + |

For the fries hatched from eggs treated with a solvent (water containing 0.1% DMSO) in the control group, the expression of the female-specific gene, Gene 5 was observed only for the genotypic females, and not for the genotypic males. Thus, the genotypic sex was consistent with the phenotypic sex. On the other hand, for the fries hatched from eggs treated with 1 ppb 17 β-estradiol, the expression of Gene 5 was observed for two out of the five genotypic males. Furthermore, the expression of Gene 6 was not observed for two out of the five genotypic females. These results show that the sexual differentiation was disrupted.

### INDUSTRIAL APPLICABILITY

The present invention provides medaka genes expressed in a female-specific manner depending on the phenotypic sex for the first time. The present invention provides a rapid and convenient method for determining the phenotypic sex using the expression of the gene as an index. The phenotypic sex can be determined within five days after hatching according to the method of the present invention. Conventionally, the phenotypic sex could not be determined until one month after hatching or the like. In addition, determination can be carried out for a number of test samples in a short time according to the method of the preset invention.

A sexual differentiation-disrupting activity of a sample can be determined rapidly and conveniently by administering a sample suspected to have a sexual differentiation-disrupting activity to a medaka, determining the phenotypic sex of the medaka according to the method of the present invention and comparing the result of the determination with the genotypic sex.

Sequence Listing Free Text

SEQ ID NO: 1: cDNA for gene 1

SEQ ID NO: 2: cDNA for gene 2

SEQ ID NO: 3: cDNA for gene 3

SEQ ID NO: 4: cDNA for gene 4

SEQ ID NO: 5: cDNA for gene 5

SEQ ID NO: 6: cDNA for gene 6

SEQ ID NO: 7: cDNA for gene 7

SEQ ID NO: 8: cDNA for gene 8

SEQ ID NO: 9: cDNA for gene 9

SEQ ID NO: 10: cDNA for gene 10

SEQ ID NO: 11: cDNA for gene 11

SEQ ID NO: 12: cDNA for gene 12

SEQ ID NO: 13: cDNA for gene 13

SEQ ID NO: 14: cDNA for gene 14

SEQ ID NO: 15: cDNA for gene 15

SEQ ID NO: 16: cDNA for gene 16

SEQ ID NO: 17: cDNA for gene 17

SEQ ID NO: 18: cDNA for gene 18

SEQ ID NO: 19: cDNA for gene 19

SEQ ID NO: 20: cDNA for gene 20

SEQ ID NO: 21: cDNA for gene 21

SEQ ID NO: 22: Genomic DNA for gene 1

SEQ ID NO: 23: Genomic DNA for gene 8

SEQ ID NO: 24: PCR primer 863.3

SEQ ID NO: 25: PCR primer 863.1

SEQ ID NO: 26: PCR primer 1/15

SEQ ID NO: 27: PCR primer 6a

SEQ ID NO: 28: PCR primer 6b

SEQ ID NO: 29: PCR primer 8.3

SEQ ID NO: 30: PCR primer F1

SEQ ID NO: 31: PCR primer R1

SEQ ID NO: 32: PCR primer F2

SEQ ID NO: 33: PCR primer R2

1. A medaka gene expressed in a female-specific manner depending on its phenotypic sex, which has a nucleotide sequence selected from the group consisting of the nucleotide sequences of SEQ ID NOS: 1 to 21.

2. A gene expressed in a female-specific manner depending on its phenotypic sex, which hybridizes with the gene defined by claim 1 under stringent conditions.

3. A gene expressed in a female-specific manner depending on its phenotypic sex, which has a nucleotide sequence selected from the group consisting of the nucleotide sequences of SEQ ID NOS: 1 to 21 as well as an intron or introns being inserted.

4. The gene according to claim 3, which has a nucleotide sequence of SEQ ID NO: 22 or 23.

5. A method for assessing a sexual differentiation-disrupting activity of a sample, the method comprising:
(1) administering a sample to be assessed for its sexual differentiation-disrupting activity to a medaka;
(2) determining the genotypic sex of the medaka;
(3) determining the phenotypic sex of the medaka based on the expression of a female-specific gene; and
(4) determining if the sexual differentiation is disrupted based on the results of steps (2) and (3).

6. The method according to claim 5, wherein the genotypic sex is determined using an egg of medaka before hatching or a fry of medaka within five days after hatching.

7. The method according to claim 5 or 6, wherein the phenotypic sex is determined using a fry of medaka within five days after hatching.

8. The method according to any one of claims 5 to 7, wherein the sample is administered to an egg of medaka before hatching or a fry of medaka within five days after hatching.

9. The method according to any one of claims 5 to 8, wherein a medaka strain of which the genotypic sex is linked to pigment expression is used.

10. The method according to any one of claims 5 to 9, wherein the phenotypic sex of the medaka is determined by examining the expression of the gene defined by any one of claims 1 to 4.

11. The method according to claim 10, wherein the expression of the gene is examined using the generation of the mRNA for the gene as an index.

12. A method for detecting an endocrine disrupter, comprising assessing a sexual differentiation-disrupting activity by the method defined by any one of claims 5 to 11.

13. An oligonucleotide for detecting the gene defined by any one of claims 1 to 4.

14. A kit for assessing a sexual differentiation-disrupting activity by the method defined by any one of claims 5 to 11, which contains the oligonucleotide defined by claim 13.

15. A kit for detecting an endocrine disrupter by the method defined by claim 12, which contains the oligonucleotide defined by claim 13.
